(19) United States
(12) Patent Application Publication
(10) Pub. No.: US 2002/0012926 A1
(43) Pub. Date: Jan. 31, 2002

(54) Combinatorial array for nucleic acid analysis

(75) Inventors: Stephen R. Quake, San Marino, CA (US); Robert Michael Van Dam, Pasadena, CA (US)

Correspondence Address: DARBY & DARBY P.C., 805 Third Avenue, New York, NY, 10022, US

(73) Assignee: Mycometrix, Inc.

(21) Appl. No.: 09/800,435

(22) Filed: Mar. 5, 2001

Related U.S. Application Data
(60) Provisional application No. 60/186,765, filed on Mar. 3, 2000.

(51) Int. Cl. C12Q 1/68; G06F 19/00; G01N 33/48; G01N 33/50

(52) U.S. Cl. 435/6; 702/20

(57) ABSTRACT

This invention relates to an array, including a universal micro-array, for the analysis of nucleic acids, such as DNA. The devices and methods of the invention can be used for identifying gene expression patterns in any organism. More specifically, all possible oligonucleotides (n-mers) necessary for the identification of gene expression patterns are synthesized. According to the invention, n is large enough to give the specificity to uniquely identify the expression pattern of each gene in an organism of interest, and is small enough that the method and device can be easily and efficiently practiced and made. The invention provides a method of analyzing molecules, such as polynucleotides (e.g., DNA), by measuring the signal of an optically-detectable (e.g., fluorescent, ultraviolet, radioactive or color change) reporter associated with the molecules. In a polynucleotide analysis device according to the invention, levels of gene expression are correlated to a signal from an optically-detectable (e.g. fluorescent) reporter associated with a hybridized polynucleotide. The invention includes an algorithm and method to interpret data derived from a micro-array or other device, including techniques to decode or deconvolve potentially ambiguous signals into unambiguous or reliable gene expression data.

This application claims priority under 35 U.S.C. § 119(e) to copending U.S. Provisional Patent Application Ser. No. 60/186,765 filed on Mar. 3, 2000, which is incorporated herein by reference in its entirety.

Numerous references, including patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification and in the priority, including all issued patents, patent applications (published or unpublished) and non-patent publications, are incorporated herein by reference in their entirety and to the same extent as if each reference was individually incorporated by reference. Many of the references cited herein are referred to numerically. A complete citation for each of these references is provided in the Bibliography appended below.

### 1. FIELD OF THE INVENTION

This invention relates in general to an array, including a universal array, for the analysis of nucleic acids, such as DNA. The devices and methods of the invention can be used for identifying gene expression patterns in any organism. More specifically, the universal arrays of the invention comprise oligonucleotide probes of all possible oligonucleotide sequences having a specified length n that may be selected by a user. The invention also relates to analytical methods which can be used to analyze data (e.g., hybridization data) from such arrays.

Applicants have discovered that values of n may be selected which are large enough to provide specificity required to uniquely identify the expression pattern of each gene in an organism of interest, and yet is also small enough that a universal microarray can be easily and inexpensively made and data therefrom can be easily and efficiently analyzed. The invention therefore also provides methods which can be used to select appropriate values of n, e.g., during the design and/or manufacture of a universal array.

The invention further relates to and provides methods of analyzing molecules, such as polynucleotides (e.g., DNA), by measuring the signal of an optically-detectable (e.g., fluorescent, ultraviolet, radioactive or color change) reporter associated with the molecules. In a polynucleotide analysis device according to the invention, levels of gene expression are correlated to a signal from an optically-detectable (e.g. fluorescent) reporter associated with a hybridized polynucleotide. A particular advantage of universal arrays of the invention is that they can be used for different genes from different organisms. It is not necessary to custom-design each chip for each application. Thus, the invention includes an algorithm and method to interpret data derived from a micro-array or other device, including techniques to decode or deconvolve potentially ambiguous signals into unambiguous or reliable gene expression data.

The invention includes nucleic acid microarrays which are typically solid surface or substrates with arrays or matrices of nucleic acid sequences that are complementary to, and therefore capable of hybridizing to, one or more nucleic acid molecules, e.g., in a sample. The arrays are preferably “addressable” arrays in which the nucleic acid sequences or “probes” are arranged at specific positions on the susbstrate, and its behavior in response to stimuli can be evaluated. For example, hybridization of a nucleic acid molecule (e.g., from a sample) to a specific probe may be detected by detecting the signal of a detectable reporter associated with that nucleic acid molecule at a specified location on the array.

In preferred embodiments, the nucleic acid molecules in the sample may correspond to one or more genes (e.g., from a cell or organism of interest). Thus, nucleic acid microarrays of the invention are useful for evaluating gene expression levels. For example, a nucleic acid micro-array may be used as a kind of “lab-on-a-chip” to identify which genes of an organism are expressed or suppressed (turned on or off) in a cell or tissue, and to what degree, under various conditions. This information can be used, for example, to study the impact of a drug on a gene, gene product (e.g. a protein or polypeptide implicated in a disease), or on a cell or organism of interest. Drug efficacy and toxicity testing are among the many uses for these techniques.

The devices and methods of the invention may be used in combination with a variety of other conventional techniques, including gel electrophoresis, polymerase chain reaction (PCR) and reverse transcription to name a few. The invention may also be implemented using microfluidic and microfabricated chip technologies.

### 2. BACKGROUND OF THE INVENTION

There are two main methodologies currently used for the construction of DNA microarrays for measuring gene expression [3, 15, 19, 13], sequencing DNA [5], or studying DNA binding proteins [2]. The first technique uses robotic fountain pens or other mechanized fluidics to “spot down” cDNA clones on a micro-array substrate. See e.g. Published PCT Application No. WO9936760 [26] and Brown et al., U.S. Pat. No. 5,807,522 [28]. This has the advantage of being flexible and requiring only simple mechanical equipment. However, the technique has disadvantages in that it is necessary to construct a cDNA library representing all the genes of interest; a time-consuming, labor intensive and expensive process. Furthermore, the practical limit for the number of genes that can be incorporated into such nucleic acid microarrays is 10,000-30,000 genes per square inch.

A second method for making nucleic acid arrays involves chemically synthesize oligonucleotides directly on a substrate. Methods and devices of this kind are disclosed, for example, in U.S. Pat. Nos. 5,922,591 and 5,143,854 and in Fodor et al., Science, 251: 767-777 (1991) [23-25]. In these systems, a photosensitive solid support or substrate is illuminated through a photolithographic mask. A selected nucleotide, typically with a photosensitive protecting group, is exposed to the substrate and binds where the substrate was exposed to light. Successive rounds of illumination through additional masks with additional nucleotides are repeated until the desired products are made. This approach requires a relatively large overhead because a new mask set must be designed and purchased for each new chip design, and the fabrication plant must be set up for large-scale production. A further disadvantage is that design of the mask set (i.e. the oligonucleotide sequences) requires a significant amount of prior knowledge of the organisms under study and expensive software tools to design the most selective oligonucleotides. The yield of oligonucleotides using light directed synthesis is extremely low, only 5% of oligonucleotides being synthesized to full length. The current demonstrated density for such arrays is roughly 100,000 oligonucleotides per square inch.

Other systems use ink-jet technology to “print” reagents (e.g., for the synthesis of nucleic acid probes) down in spots on the solid surface of an array. These arrays may provide a higher chemical yield than other known methods. However, the printing procedure is a difficult serial process because the density of spots is low and is different for each gene of each organism of interest.

In summary, the disadvantages of previous DNA micro-array devices include: (1) a high cost per array; (2) limitations regarding specificity (e.g., each chip is specially designed to study one organism or tissue); and (3) a need to design and manufacture a new chip when new genes are discovered in the organism of interest.

It is thus desirable to provide an adaptable or universal chip which can be used for the analysis of gene expression in any organism, e.g. from prokaryotes to humans.

### 3. SUMMARY OF THE INVENTION

The invention provides a method and an array device for the analysis of DNA or other molecules, including a universal array, e.g. for combinatorial chemistry or DNA analysis.

An object of the present invention is to identify gene expression patterns in any organism with one device, e.g. with minor modifications to a universal device which can replace conventional DNA micro-arrays in any application.

An additional object of the present invention is to provide an automated DNA analysis assay.

A further object of the present invention is to provide a kit for detecting gene expression patterns in any organism.

A further object of the invention is to provide a universal micro-array; i.e., an array of oligonucleotides having a specified sequence length n (referred to herein as “n-mers”) wherein all possible nucleotide sequence of length n are present on the array. Current technologies use chips having only certain specific oligonucleotides that are carefully selected to detect particular genes. Thus, for every organism (or even for different cells from the same organism that express different genes) it is necessary to design a new micro-array. The universal arrays of this invention therefore offer the advantage of being useful for studying gene expression in any cell or organism; thereby making a specially designed chip unnecessary.

Still another object of the invention is to determine and provide useful values for the oligonucleotide sequence length n that may be used in a universal array, particularly for preferred embodiments of analyzing gene expression.

Additional objects of the invention include measuring gene expression levels, sequencing nucleic acids (e.g., DNA), “fingerprinting” DNA and other nucleotide sequences, measuring interactions of proteins and other molecules with nucleic acid sequences (e.g., with all oligonucleotides of a specified length n), and detection of mutations and polymorphisms including single nucleotide polymorphisms (SNPs).

Yet another object of the invention is to provide algorithms for analyzing data from an array of all posible n-mers; e.g., to solve for gene expression levels in a nucleic acid sample.

Other objectives will be apparent to persons of skill in the art.

In accomplishing these and other objectives, the invention provides algorithms for decoding and/or deconvoluting potentially ambiguous hybridization data and thereby provide meaningful information, e.g., regarding gene expression levels in a cell or organism (or, more typically, in a sample of nucleic acids obtained from a cell or organism). In such algorithms, both expression levels for a plurality of genes (e.g., for individual genes in a genome) and levels of hybridization to a plurality of oligonucleotide probes (e.g., on a microarray) may be represented as vectors (referred to as “expression vectors” and “hybridization vectors”, respectively). Hybridization of the genes to the different probes may be represented as a mathematical “mapping” of an expression vector to a hybridization vector. The algorithms of the invention use an improved and efficient process for solving linear equations associated with such a mapping, by identifying subblocks of probes and genes in which the oligonucleotide probes in each subblock collectively hybridize to all of the genes in the subblock, and do not hybridize to any gene not in the subblock. By identifying the smallest possible subblocks for a particular collection of genes or nucleic acids (e.g., for a particular genome), the collection of linear equations associated with a particular hybridization experiment is reduced or “projected” to sets of simpler linear equations, each set representing the hybridization of a smaller number of genes to a few specific probes on the microarray. These sets of linear equations can then be easily and efficiently solved to reliably determine gene expression levels.

The invention is based in part on the inventors' discovery that appropriate probe lengths n may be selected that are small enough that fabrication of universal micr-arrays comprising all oligonucleotide probe sequence of length n is feasible and average probe “degeneracy” is low (i.e., each probe only hybridizes to, on average, only a few nucleic acids or genes). As a result, a hybridization matrix describing the “mapping” of expression levels to hybridization data in an experiment may be easily deconvoluted using the algorithms of the invention to identify relatively small subblocks.

A statistical model for determining average probe degeneracy is also provided, and this model may be used, e.g., to select an appropriate probe length n for a universal array that achieves an average probe degeneracy value appropriate for analyzing a nucleic acid sample (e.g., of genes from a particular genome) using a universal array of probe length n. Using this model, predictions were made of the parameter values (e.g., n-mer size) needed to achieve an average degeneracy of 1. A degeneracy of 1 represents an ideal or trivial case of degeneracy or signal confusion, and is therefore particularly desirable. Further calculations with actual genomic data indicate that the predicted parameter values ensure that most subblocks have size 1, demonstrating correspondence between predicted and actual calculated or determined expression levels. Preferably, the average degeneracy value of probes used in the analytical methods of this invention will be less than about ten. For example, in other preferred embodiments of the invention, n values may be selected for a universal array so that the average probe degeneracy, when used to analyze a particular collection of nucleic acids (e.g., a particular genome) will be about 2, about 3, about 4 or about 5.

Polynucleotides are hybridized on a substrate, and a hybridization signal is produced, for example, according to a reporter or label associated with the polynucleotide, such as a fluorescent marker. Alternatively, complementary polynucleotides can be post-stained with an intercalating dye. Another variation is to use affinity purification to pull down the fragment of interest, i.e., using biotinylated oligonucleotides and streptavidin coated magnetic beads (e.g., for enrichment and normalization to enhance an RNA population). Thus, the invention can be used in combination with a variety of techniques, including any hybridization techniques, such as any micro-array technology. This includes the the pen-spotting arrays, light sensitive masks, and ink jet devices described herein. Devices of the invention also include microfabricated and microfluidic devices. In preferred embodiments, the substrate of the micro-array is planar and contains a microfluidic chip made, e.g., from a silicone elastomer impression of an etched silicon wafer according replica methods in soft-lithography. See, e.g., the devices and methods described in pending U.S. patent application Ser. No. 08/932,774 (filed Sep. 25, 1997) and Ser. No. 09/325,667 (filed May 21, 1999), and in International Patent Publication No. WO 99/61888. See also, U.S. provisional patent application Ser. No. 60/108,894 (filed Nov. 17, 1998) and No. 60/086,394 (filed May 22, 1998). These methods and devices can further be used in combination with the methods and devices described in pending U.S. provisional application Ser. No. 60/141,503 (filed Jun. 28, 1999); No. 60/147,199 (filed Aug. 3, 1999) and No. 60/186,856 (filed Mar. 3, 2000).

In preferred embodiments, the microfabricated devices and algorithms of this invention may be used for the identification of gene expression patterns of genes from the genome of a higher eukaryotic organism, including genes from the genome of a mammalian organism such as a mouse or a human. However, the algorithms and microarrays of the invention can be used to evaluate any nucleic acid sample, including nucleic acid sample that comprise genes from the genome of any organism (including viral genomes, bacterial genomes such as the E. coli genome, and the genomes of lower eucaryotes such as the yeast S. cerevisiae and S. pompe). The universal array is fast and requires only small amounts of material, yet provides a high sensitivity, accuracy and reliability.

### 4. BRIEF DESCRIPTION OF THE DRAWINGS

FIG. 1 shows the comparison of measurements and predictions of average degeneracy (λ) for yeast DNA assuming single-base mismatches are allowed. Continuous lines represent predictions of average degeneracy from the theoretical model presented in Example 3 infra and as a function of the oligonucleotide sequence length n for various levels of transcript length truncation L. Discrete points represent actual values determined from in silico analysis of sequences in the yeast genome.

FIG. 2 shows the comparison of measurements and predictions of average degeneracy (λ) for mouse DNA assuming single-base mismatches are allowed. Continuous lines represent predictions of average degeneracy from the theoretical model presented in Example 3 infra and as a function of the oligonucleotide sequence length n for various levels of transcript length truncation L. Discrete points represent actual values determined from in silico analysis of sequences in the yeast geneome.

FIG. 3 shows the relationship between the oligonucleotide sequence length n and truncation length such that the average degeneracy, λ is one.

FIGS. 4A-B show the distribution of transcript lengths for yeast ORFs (FIG. 4A) and the mouse Unigene database (FIG. 4B). To clearly show the distribution shapes, the longest genes have been omitted from each plot. The length distribution of the yeast ORFs has been fit to a generalized exponential function with the form:

$$f ( x ; {λ}_{0} , {n}_{0} ) = \frac{1}{{λ}_{0}} {( \frac{x}{{λ}_{0}} )}^{{n}_{0}} {ⅇ}^{- x / {λ}_{0}} ,$$

and this fit is indicated by the dark solid line in FIG. 4A.

FIGS. 5A-J shows the fit of degeneracy histograms generated in silico from yeast genomic sequences (▪) with predictions from the analytical model described in Example 3 infra (dark solid lines). Each histogram shows the relative number of oligonucleotide probes of a specified length n having a given degeneracy value for a particular number m of tolerated base-pair mismatches: FIG. 5A, n=8 and m=0; FIG. 5B, n=8 and m=1; FIG. 5C, n=9 and m=0; FIG. 5D, n=9 and m=1; FIG. 5E, n=10 and m=0; FIG. 5F, n=10 and m=1; FIG. 5G, n=11 and m=0; FIG. 5H, n=11 and m=1; FIG. 5I, n=12 and m=0; FIG. 5J, n=12 and m=1.

FIGS. 6A-H show histograms of minimum degeneracy values of mouse genes for oligonucleotide probes having a sequence length n=11 or 12, allowing for hybridization with as much as one base-pair mismatch (i.e., m=1). Histograms were generated in silico, as described in Example 3 and using sequences from the mouse Unigene databank that were either full length (i.e., untruncated) or were truncated in silico to a fixed length L. FIG. 6A, n=11 and L=50; FIG. 6B, n=11 and L=100; FIG. 6C, n=11 and L=200; FIG. 6D, n=11 and L=“untruncated”; FIG. 6E, n=12 and L=50; FIG. 6F, n=12 and L=100; FIG. 6G, n=12 and L=200; FIG. 6H, n=12 and L=“untruncated”.

FIGS. 7A-B show fractions of oligonucleotide sequences having a specified length n that are uniquely present (with a mismatch tolerance m=1) in collections of sequences from the yeast (FIG. 7A) and mouse (FIG. 7B) genomes. The fractions of unique oligonucleotide sequences were determined for each values of n from raw sequences (♦) obtained from genome databases, as well as for sequences that were truncated in silico to fixed length L of 50 (▪), 100 (▴) and 200 (●) bases.

### 5. DETAILED DESCRIPTION OF THE INVENTION

### 5.1. Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

General Definitions.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The term “substantially pure” indicates the highest degree of purity which can be achieved using conventional purification techniques known in the art.

A “sample” as used herein refers to a material which can be tested, e.g., for the presence of a polymer (for example, a particular protein or nucleic acid) or for a particular activity or other property associated with a polymer (e.g., a catalytic or binding activity associated with a particular polypeptide).

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “molecule” means any distinct or distinguishable structural unit of matter comprising one or more atoms, and includes, for example, polypeptides and polynucleotides.

Molecular Biology Definitions.

In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning. A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

The term “polymer” means any substance or compound that is composed of two or more building blocks (‘mers’) that are repetitively linked together. For example, a “dimer” is a compound in which two building blocks have been joined togther; a “trimer” is a compound in which three building blocks have been joined together; etc. The individual building blocks of a polymer are also referred to herein as “residues”.

A “biopolymer”, as the term is used herein, is any polymer that is produced by a cell. Preferred biopolymers include, but are not limited to, polynucleotides, polypeptides and polysaccharides.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil. Polynucleotides of the invention may also comprise any of the synthetic or modified bases described infra for oligonucleotide sequences.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage.

The polynucleotides herein may also be modified with a label or reporter capable of providing a detectable signal, either directly or indirectly. The terms “label” and “reporter” are used synonymously herein, and refer to any molecule, or a portion thereof, that provides a detectable signal (either directly or indirectly). The reporters and labels used in the present invention are generally capable of associating with or of being associated with a molecule (such as a polynucleotide or protein) to permit identification of the molecule. A reporter may also permit determination of certain characteristics of a molecule such as size, molecular weight, or the presence or absence of certain constituents or moieties (such as particular nucleic acid sequences or particular restriction sites). Exemplary reporters includes dyes, fluorescent, ultraviolet and chemiluminescent agents, chromophores and radio-labels. Particularly preferred reporters include Cy3, CyS, fluoroscein and phycoerythrin, as well as other reporters identified in this specification.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

“Amplification” of a polynucleotide, as used herein, denotes the use of polymerase chain reaction (PCR) to increase the concentration of a particular DNA sequence within a mixture of DNA sequences. For a description of PCR see Saiki et al., Science 1988, 239:487.

“Chemical sequencing” of DNA denotes methods such as that of Maxam and Gilbert (Maxam-Gilbert sequencing; see Maxam & Gilbert, Proc. Natl. Acad. Sci. U.S.A. 1977, 74:560), in which DNA is cleaved using individual base-specific reactions.

“Enzymatic sequencing” of DNA denotes methods such as that of Sanger (Sanger et al., Proc. Natl. Acad. Sci. U.S.A. 1977, 74:5463) and variations thereof well known in the art, in a single-stranded DNA is copied and randomly terminated using DNA polymerase.

A “gene” is a sequence of nucleotides which code for a functional “gene product”. Generally, a gene product is a functional protein. However, a gene product can also be another type of molecule in a cell, such as an RNA (e.g., a tRNA or a rRNA). For the purposes of the present invention, a gene product also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “genome” is used herein to refer to any collection of genes or, more generally, gene sequences (for example, transcripts of genes such as mRNA, cDNA derived therefrom, or cRNA derived therefrom). Thus, in one embodiment a genome may refer to a collection of chromosomal nucleic acid sequence, e.g., from a cell or organism, which corresponds to all of the genes of that cell or organism. Alternatively, the term genome is also used herein to refer to nucleic acid sequences that correspond to a particular subset of a cell or organism's genes. For example, in preferred embodiments the devices and methods of this invention may be used to determine which genes are expressed by a particular cell or organism (e.g., under certain conditions of interest to a user). Therefore, the term genome, as it is used to describe the present invention, may also refer to a collection of genes or gene transcripts that are or may be expressed by a cell or organism.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also be said to be “expressed” by the cell.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label or reporter, such as biotin or a fluorescent dye (for example, Cy3 or Cy5) has been covalently conjugated. Oligonucleotides therefore have many practical uses that are well known in the art. For example, a labeled oligonucleotide can be used as a probe to detect the presence of a nucleic acid. Oligonucleotides (one or both of which may be labeled) can also be used as PCR primers. In a further embodiment, an oligonucleotide of the invention can form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

An “antisense nucleic acid” is a single stranded nucleic acid molecule which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, triple helix interactions, ribozymes and RNase-H mediated arrest. Antisense nucleic acid molecules can be encoded by a recombinant gene for expression in a cell (e.g., U.S. Pat. No. 5,814,500; U.S. Pat. No. 5,811,234), or alternatively they can be prepared synthetically (e.g., U.S. Pat. No. 5,780,607).

Specific non-limiting examples of synthetic oligonucleotides envisioned for this invention include, in addition to the nucleic acid moieties described above, oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497, 1991). Other synthetic oligonucleotides may contain substituted sugar moieties comprising one of the following at the 2′ position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O-; S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substitued silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. Conditions of appropriate stringency may be readily determined by a skilled artisan, e.g., using semi-empirical formulas to determine nucleic acid duplex stability [1].

For preliminary screening for homologous nucleic acids, low stringency hybridization conditions, corresponding to a Tₘ (melting temperature) of 55° C., can be used, e.g., 5× SSC, 0.1% SDS, 0.25% milk, and no formamide; or 30% formamide, 5× SSC, 0.5% SDS). Moderate stringency hybridization conditions correspond to a higher Tₘ, e.g., 40% formamide, with 5× or 6× SSC. High stringency hybridization conditions correspond to the highest Tₘ, e.g., 50% formamide, 5× or 6× SSC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2× SSC, at 42° C. in 50% formamide, 4× SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligoncucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6× SSC/0.05% sodium pyrophosphate, or other conditions that afford equivalent levels of hybridization.

### 5.2. Overview of the Invention

The invention provides devices and methods for the analysis of nucleic acids. More particularly, the analysis of gene expression patterns can be achieved by synthesizing all possible n-mers, e.g. of a gene or genome, where n is large enough that one finds the specificity to uniquely identify the expression pattern of each gene in the organism but small enough that a practical and efficient method and device can be provided.

In the microfabricated device according to the invention, levels of gene expression are correlated to a hybridization signal from an optically-detectable (e.g. fluorescent) reporter associated with the polynucleotides. These hybridization signals can be detected by any suitable means, preferably optical, and can be stored for example in a computer as a representation of gene expression levels. Universal chips according to the invention can be fabricated for not only DNA but also for other molecules such as RNA, peptide nucleic acid (PNA) and polyamide molecules [4], to name a few.

According to one aspect of the invention, a key to the identification of gene expression patterns is to find a fragment or mer-size (n) that is large enough to have useful specificity, and is small enough to be practical for implementation on a small and/or automated or high-throughput scale, including the practical manufacture of suitable analysis devices. It is known for example that a value of n=50, i.e. all possible 50-mers, would be useful for identifying gene expression patterns in a universal array device. However, the resulting number of possible combinations of nucleotides and synthesized 50-mer oligonucleotides is impractically high; specifically 4⁵⁰ ≈10³⁰ oligonucleotides. This would require a micro-array of 10¹⁵ pixels per inch to realize a one-inch chip i.e., a pixel size with sub-angstrom dimensions. Therefore, a universal array on a chip having 50-mers is clearly impractical if not impossible.

Useful information has been obtained from cDNA libraries containing all possible 8-mers, i.e. n=8, but these applications are not universal. See e.g. U.S. Pat. No. 5,525,464 [27].

In one aspect of the invention, the physical limitations of the device are calculated based on possible values of n when all n-mers may be synthesized in one square inch. The physical dimension of one square inch is an arbitrary choice, but is approximately the useful size for gene expression experiments that is compatible with existing equipment and methodologies. Any other convenient dimension may be used.

“Ink jet” printer systems and robotic fountain pen technologies can realize pixel sizes of 100 microns, which allows 60,000 distinct oligomers per square inch to be distinguished. This corresponds to n=8. Light-directed synthesis is constrained by the diffraction limit, which in the semiconductor industry is currently 0.28 microns. This corresponds to ≈8,000,000,000 distinct oligomers per square inch, or n=16. Resolution of the number of oligomers (e.g. oligonucleotide molecules) on the chip is another limiting factor. Currently the optimal resolution is about 100,000 distinct oligomers per square inch. Near field techniques [21] or electrochemical readout [10] may ultimately allow scanning of pixels down to 30 nanometers, which corresponds to 700,000,000,000 oligomers per square inch and a maximum of n=20. Within the bounds of current practical limits of lithographic chemical patterning, a minimum pixels size of 1 micron could be considered, allowing n=15 and below this the minimum useful value of n is n=10, corresponding to a pixel size of 25 microns. Preferred universal combinatorial arrays of the present invention are provided having a range of n=10 to n=15.

Given the feasibility and existence of a universal combinatorial device with a range of about n=10 to n=15, an algorithm is described to interpret the data from a device of this scale and using oligomers in this size range. The algorithm is useful for decoding or deconvolving the potentially degenerate or ambiguous hybridization signals from oligomers of this size into unambiguous and/or accurate (e.g. statistically reliable) gene expression data. The techniques of the invention are particularly useful in circumstances where oligomers of less than n≈15 may not be sufficiently specific for the desired assay. That is, larger oligomers (e.g. n=50) are generally sufficiently specific, but are impractical or impossible to work with. Shorter oligomers are more practical, for example in size, scale and number, but may not be sufficiently specific. The invention provides techniques whereby shorter and more practical oligomers can be used to provide sufficiently specific results.

Among the advantages of the invention are that multiple experiments can be achieved with a particular molecular species, whereby for example oligonucletides and oligonucleotide groups can be predicited to correspond to particular genes without prior knowledge of sequence data. That is, the invention can be used when sequence information is known (as in the Examples infra), and such information can serve to verify the techniques described herein. However, the invention is more general and does not require knowledge of a particular genome. For example, by performing multiple experiments instead of just one it is possible to determine gene expression levels without knowing the genome sequence beforehand.

Another advantage of the predictive approach is that experimental data can be re-analyzed as more genomic data is accumulated, thus removing the need to repeat experiments.

Still another advantage of the invention is that, unlike techniques using conventional micro-arrays, it is not necessary to design and manufacture a whole new to chip in order to study a newly discovered gene.

### 6. EXAMPLES

The present invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### 6.1. Example 1

### Genetic Analysis with a Universal Array

This Example describes the theoretical correlation between the optical signals generated during hybridization experiments, to gene expression levels in the mouse and yeast genome.

Notation.

The genome is represented as a set, G, and its constituent nucleic acid sequences is represented as G={g1, g2, . . . , gN_{g}}. N_{g} is the total number of genes. Each sequence called here a “gene” corresponds to one mRNA sequence which may be found in the cell. (The mRNA is transcribed from individual genes in the DNA, and serves as the template from which the cell makes proteins. The amount of each particular mRNA sequence in the cell reflects the expression level of the corresponding gene.) At any given instant (and under a given set of experimental conditions), the expression level of the genes in a sample can be represented as a single N_{g}-dimensional vector in expression-level-space (ε),

E=(E₁,E₂, . . . , Eⱼ, . . . , E_{N}_{_{g}})^{T},

in which the superscript T denotes the transpose vector (i.e., indicating that the vector E may preferably be written as a column vector rather than as a row vector). Each element of the vector, Eⱼ, is a real quantity, equal to the expression level of genes gⱼ. These are the unknown quantities in a hybridization experiment.

The universal array of the present invention consists of a regular pattern of distinct spots of DNA sequences, each spot containing oligonucleotide strands of length n. In the set

O(N)={o₁, o₂, . . . , oₗ, . . . , o_{N}_{ₒ}}

of all possible sequences of length n, there are Nₒ=4ⁿ members, and all of these are represented on the array. Therefore there is a one-to-one mapping between the position of a spot on the array and its corresponding oligonucleotide sequence.

During an exemplary hybridization experiment, molecules of fluorescently or radioactively labeled mRNA from a sample of interest are mixed with the n-mer array under specific conditions. The duplexes that form between the sample and the complementary oligonucleotide each correspond to a spot or hybridization signal, which is related to the total amount of mRNA from several different genes. The hybridization signal intensities can be represented as an Nₒ-dimensional vector in hybridization-signal-space (S), where

S=(S₁, S₂, . . . , Sᵢ, . . . , S_{N}_{ₒ})^{T}

As explained supra for the expression vector E, the superscript T denotes the transpose (i.e., indicating that the vector S may also preferably be written as a column vector). Each element Sᵢ is a real quantity equal to the hybridization signal intensity for oligonucleotide oᵢ. In general, the observed hybridization signal for each oligonucleotide depends on numerous experimental parameters (e.g. time, temperature, reaction conditions, etc.). It is estimated however that the observed hybridization signal is linearly related to the number of complementary mRNA molecules, which is accurate for labeling schemes in which one label is attached to each mRNA molecule.

In schemes where the amount of incorporated label depends on the strand length, a minor modification is needed. The linear coefficients (for multiplying the expression level of each gene) must be divided by the gene length. (These coefficients constitute the affinity matrix, H). Note also that the estimation that the hybridization signal is linearly related to the number of complementary mRNA molecules is not expected to hold under conditions of “saturation”. Saturation occurs when all of the oligonucleotide molecules tethered to one spot on the n-mer array have captured a strand of mRNA, and therefore no more mRNA binding can occur at that spot. Saturation conditions place a physical limit on the maximum hybridization signal that can be observed, because of the introduction of non-linearities for n-mers which are complementary to a large number of gene sequences. However, this can be overcome easily by scanning through the gene sequences and removing them from consideration, since they provide no useful information. This is not necessary in preferred embodiments of the present invention, because the algorithm of the invention automatically eliminates these n-mers by looking first for the least ambiguous spots. According to this approach, the estimate of linear correspondence holds true.

The hybridization experiments can be considered to be a type of mathematical mapping, H: ε→S, from the space of expression levels, e, to the space of hybridization signals, S. Representing this mapping with a matrix, H, a hybridization experiment can be described by the following equation:

$$\begin{matrix}S \\ ( {N}_{o} x1 )\end{matrix} = \begin{matrix}H \\ ( {N}_{o} {xN}_{g} )\end{matrix} \cdot \begin{matrix}E \\ ( {N}_{g} x1 )\end{matrix} \qquad ( 1 )$$

where the relevant dimensions have been given beneath each vector and matrix. Each entry, Hᵢⱼ, of the hybridization matrix represents the affinity with which gene gⱼ binds to oligonucleotide, oᵢ (i.e., the “stickiness” of the interaction). It also includes an overall scale factor relating a specific quantity of hybridized DNA to the corresponding hybridization signal.

The affinities depend on the general hybridization conditions (such as temperature, salt concentration, pH, solvent), and the nucleotide sequences of molecules i and j. Several semi-empirical formulae have been published for estimating these values with reasonable accuracy. See e.g. [1]. Hybridization experiments can also be achieved with known amounts of mRNA (or other nucleic acids) thus allowing deduction of the affinities of the mRNA from the resulting hybridization patterns directly.

Solving Gene Expression Levels.

Given the vector of known hybridization signals, S, and the matrix of known binding affinities, H, the next objective is to solve the unknown vector of gene expression levels, E. A matrix equation can be written to represent a system of Nₒ linear equations for these N_{g} unknowns:

$$\begin{matrix}{S}_{1} = & {H}_{11} {E}_{1} & + & {H}_{12} {E}_{2} & + & … & + & {H}_{1 {N}_{g}} {E}_{{N}_{g}} \\ {S}_{2} = & {H}_{21} {E}_{1} & + & {H}_{22} {E}_{2} & + & … & + & {H}_{2 {N}_{g}} {E}_{{N}_{g}} \\ ⋮ & ⋮ & & ⋮ & & & & ⋮ \\ {S}_{{N}_{o}} = & {H}_{{N}_{o} 1} {E}_{1} & + & {H}_{{N}_{o} 2} {E}_{2} & + & … & + & {H}_{{N}_{o} {N}_{g}} {E}_{{N}_{g}}\end{matrix} \qquad ( 2 )$$

This system is not invertible because generally Nₒ>N_{g} and therefore H is not square and does not have an inverse.

A strategy therefore has been devised for solving the unknown vector of gene expression levels efficiently. The first part of the strategy begins with a reduction in the dimensionality of H, reducing it to a matrix H′ with only N_{g} rows. To do so, subsets of size N_{g}, O′(N) are considered and a projection P: O(N)→O′(N) is sought, such that the projected matrix H′=P·H is invertible. The expression levels may then be solved by the relation:

E=(H′)⁻¹·S′ (3)

where S′ is the projection of the hybridization signal vector, P·S. Generally Nₒ>>N_{g}, so that there is a considerable reduction in dimensionality and therefore considerable freedom in choosing a projection.

The second part of the strategy is to take advantage of this flexibility to make Equation (3) as easy to solve as possible. The inversion of a general N_{g}×N_{g} matrix is computationally difficult (For some organisms of interest, such as human beings, N_{g} may be on the order of 10⁵), but the complexity of inversion can be drastically reduced by selecting a projection which results in a block diagonal form for H′. In block diagonal form, the problem of inverting a large matrix is converted to several inversions of smaller matrices (the “blocks”). If these blocks are small or very small, then the inversion is easy. In fact, if the block size is unity (one), the matrix is diagonal, and the inverse is trivial: the reciprocal of each element is taken. Example 2 describes a relatively simple algorithm which minimizes the size of the blocks in the projected matrix.

It should be noted that the approach of selecting only a subspace of O(N) may ignore some of the information contained in the hybridization signals. However, by choosing a projector with the above properties, the most ambiguous information in the n-mer array tends to be ignored.

In theory, for a given size of n-mer array, n, it is only necessary to compute the projection, P, once. If, in addition, all hybridizations are performed under similar sets of conditions, then computation of affinity matrix H and the related matrix H′ can be achieved ahead of time. When a hybridization is performed, the signal vector S is measured and is projected by P. Then the expression levels are easily solved by carrying out the matrix multiplication (H′ is block diagonal) in Equation (3).

Factors Affecting Computational Tractability.

The likelihood of finding a projector with the properties described above increases with the sparseness of the affinity matrix H. Consider first a single row of H. The non-zero entries in this row correspond to genes for which oligonucleotide oᵢ has significant binding affinity. (The assumption is made regarding non-zero entries that a cutoff value of m is defined such that pairs of sequences containing more than m mismatches have exactly zero binding affinity). The number of non-zero entries in a row corresponds to the “degeneracy” of the corresponding oligonucleotide. Furthermore the degeneracy of an oligonucleotide is the number of genes that have a significant contribution to the hybridization signal. If the average degeneracy is low, then the matrix would be sparse.

It can be expected that the average degeneracy decreases as the array size (n) increases because it becomes less likely that a given n-mer can occur in several different genes. The average degeneracy also depends on a particular genome. As the genome size increases, the incidence of length n sequences contained within it increases. Therefore, the probability that a particular sequence occurs multiple times in the genome increases, as does the average degeneracy.

In certain embodiments the average transcript length may be decreased. For example, nucleic acids in a sample may be incubated with a nuclease or other enzyme that digest polynucleotides, effectively truncating nucleic acids in a sample before hybridization to an n-mer array, and thereby eliminating unnecessary regions of the genomic sequence. As a particular, non-limiting example, some enzymes degrade nucleic acids, such as RNA molecules, in the 3′→5′ direction. The average length <ΔL> by which the nucleic acid is truncated is dependent upon, and can thereby be controled by, parameters of the reaction such as incubation time and temperature. Adding such an enzyme to a nucleic acid sample (e.g., a preparation of mRNA from a cell or organism) for a specific amount of time will therefore decrease the mRNA length, on average, by an amount <ΔL>. Thus, instead of looking at the entire gene sequence when computing hybridization affinities Hᵢⱼ. the last ΔL bases of each sequence may be ignored since, on average, they will not be present in the sample. (For oligonucleotides oᵢ which pair only with the digested part of gene gⱼ, the corresponding entries, Hᵢⱼ can be set to zero.). Preferred values for <ΔL> include values of less than about 500, about 100 or about 50 bases. Particularly preferred values of <ΔL> are between about 50-500 bases and, more preferably, between about 50-100 or between about 100-500 bases.

In a more preferred embodiment, single stranded nucleic acids (e.g., mRNA molecules) in a sample may be polymerized from the 3′-end for a certain amount of time such that, on average, a length of <L> bases in each nucleic acid becomes double stranded. This can be achieved by treating the nucleic acid with a suitable polymerase enzyme and primers suitable for polymerizing the nucleic acid. For example, in preferred embodiments where the nucleic acid is mRNA, a sample may be incubated with a suitable RNA polymerase and primers complementary to the poly-A sequence at the end of the transcripts. Washing, followed by treatment with a nuclease enzyme which only digest single stranded nucleic acids may then remove any portion of the nucleic acid molecules that are not double-stranded. As a result, the nucleic acids in the sample can be effectively truncated by an average length <L> that may be controlled, e.g., by controlling the conditions of the polymerization reaction (for example, conditions of time and temperature). Preferred values for an average truncated length <L> include lengths of less than about 500, about 100 or about 50 bases. Particularly preferred average truncated length values <L> are between about 50-500 bases and, more preferably, between about 50-100 or between about 100-500 bases.

Non-specific Binding (Mismatches).

It is well known in the art that binding between polynucleotide strands is not restricted to perfectly matched complementary sequences but can and does occur even between molecules which are mismatched at several bases.

As the number of allowed mismatches increases, clearly the average degeneracy will rise sharply. It is therefore important if not necessary to impose stringent conditions during hybridization to exclude the possibility of a large number of allowed mismatches. In order to achieve this goal, the hybridization conditions can be arranged so as to impose a cutoff value m representing the maximum number of allowed mismatches in any duplex between any pair of sequences. Thus any pairing of oligonucleotide oᵢ and gene gⱼ which matches perfectly at n-m positions has a corresponding non-zero entry in the affinity matrix, and any pairing where this condition is not satisfied has an entry of zero. An important consequence of this assumption is that pairs of genes and oligonucleotides which may hybridize with one another can be identified based on the sequences alone, making possible the rapid calculation of degeneracy values.

In practice, stability is not a function of the number of mismatches alone [14, 6, 18, 8]. Stability depends strongly on the positions of the mismatches within the binding region of the sequences, with internal mismatches having a much more pronounced destabilizing effect. Furthermore, duplex stability is a function of the particular nucleotides present at the matched and mismatched positions. Accordingly, a mismatch cutoff value may not be needed. In any case, techniques for reducing these inconvenient functional dependencies of stability have been reported in the literature. The simplest approaches for reducing the dependence on nucleotide identities seems to be the addition of auxiliary substances which bind in the grooves of DNA duplexes [11], or using polynucleotides other than DNA [9]. A recently reported technique for reducing position dependence is the addition of very short sequences to the hybridization mix which will decrease the relative stability of end mismatches by the phenomenon of contiguous stacking stabilization [20, 22]. Recent publications also indicate that electric fields may help to destabilize mismatches [17]. Using one or more of these techniques and other general approaches for destabilizing mismatched sequences, a mismatch threshold of m=1 or even m=0 may be achieved. For example, several hybridization schemes are currently able to detect single nucleotide variations between DNA strands [12, 7].

### 6.2. Example 2

### Algorithm for Determination of Gene Expression Patterns

In this Example an algorithm is presented for construction of the projector, P, (described in Example 1), for reducing the dimensionality of the space of oligonucleotides O(N). The algorithm is designed to find a projector which results in a nearly diagonal form for H if H is sufficiently sparse.

Definitions.

In preferred embodiments, the following quantities are used in connection with the algorithm. The quantities are, in general, functions of the particular genome considered, as well as of the parameters n and m and any enzymatic treatment which alters the sequence space covered by the transcripts.

The quantity Degen(oⱼ) refers to the degeneracy of the oligonucleotide oᵢ. The terms “degeneracy” and “ambiguity”, as they are used herein, refer to the number of different genes to which a probe having an oligonucleotide sequence of length n may hybridize. Thus, the degeneracy of an oligonucleotide probe represents the number of different nucleic acids in a sample (i.e., the number of different genes) which will contribute to the hybridization signal seen on that probe.

The quantity GeneSet(oⱼ) denotes that set of genes that can bind or hybridize to the oligonucleotide probe oⱼ. Generally, this will be the set of all genes that are complementary to the oligonucleotide sequence of oⱼ within a specified number of base pair mismatches m. This set has a size equal to Degen(oⱼ) and contains the genes corresponding to all non-zero elements of row j in the hybridization affinity matrix H. Alternatively, the GeneSet(oⱼ) may be said to contain all genes which contain the complementary sequence of oⱼ to within m mismatches.

The Oligonucleotide Set(gᵢ) refers to the set of oligonucleotides to which the gene gᵢ is able to hybridize or bind. This set corresponds to the set of all oligonucleotides which have non-zero element of column i in the hybridization affinity matrix H. A useful interpretation of this set is that it is the set of all complementary subsequences of length n which are found in the gene gᵢ (to within m mismatches).

The term “minimum degeneracy” of gene gᵢ, which is also denoted here as MinDegen(gᵢ), refers to the lowest degeneracy value of any of the oligonucleotides in Oligonucleotide Set(gᵢ) (defined supra).

The term “subblock”, as used herein, refers to a collection of oligonucleotides and genes, preferably such that the union of the GeneSet for all oligonucleotides in the subblock contains all of the genes in the subblock, and no other genes. Thus, in preferred embodiments, a subblock will contain only oligonucleotides that hybridize to genes associated with that subblock, and do not hybridize to genes that are not associated with that subblock. In preferred embodiments of the invention, the projected affinity matrix H′ will be in block diagonal form if genes are assigned to distinct subblocks that have no genes in common with one another.

In preferred embodiments, the degeneracy of an oligonucleotide and the genes which belong to the gene set may be determined by searching through the entire genome, and checking each gene to determine where the oligonucleotide exists. In a particularly preferred approach that may save a substantial amount of time, these results may be precomputed by scanning through the genome beforehand. A further preferred approach, for the optimization of memory storage, is to discard the gene set for those oligonucleotide probes having a degeneracy that is greater than some predetermined cutoff level or “threshold” T that may be selected by a user. Preferred maximum degeneracy values (which are therefore preferred threshold values) are no more than 100, no more than 50, no more than 20 or no more than 10. More preferably, the maximum degeneracy of any selected oligonucleotide (i e., the threshold value) is no more than five, more preferably no more than four, still more preferably no more than three, and even more preferably no more than two. In particularly preferred embodiments, the maximum degeneracy of any selected oligonucleotide is unity (i.e., equal to one).

Generating Subblocks.

The algorithm of this example essentially selects certain key oligonucleotides from the set of all 4ⁿ oligonucleotides, such that the corresponding subblock sizes in an array are as small as possible. If the subblock size is 1, this means that the single oligonucleotide in that subblock has a degeneracy of 1 (i.e. the oligonucleotide is a subsequence of only one gene). Further, if the subblock size is 2, this means that the two oligonucleotides in that subblock are collectively found in only two out of all the genes. When the algorithm is complete, each gene in the genome is represented in one subblock, making it possible to rearrange the order of genes and oligonucleotides such that the subblocks could be placed along the diagonal of H′.

Preferably, only “invertible” subblocks should be formed. To confirm that a subblock is invertible, it is converted into a matrix and then the determinant is computed. (If the determinant is non-zero, then the matrix is invertible). The procedure for converting a subblock into a matrix is to treat the oligonucleotides in the subblocks as the rows of the array, and the genes in the subblock as the columns in the array. The elements of the matrix are then simply taken from the corresponding entries of the affinity matrix.

The algorithm proceeds as follows:

1. Compute the minimum degeneracy (MinDegen(gⱼ)) for all genes, g_{j.}

2. Sort genes in order of increasing MinDegen(gⱼ). Placing genes in this order is a strategy for achieving a near-diagonal form for the final projected matrix since it means that the smallest possible subblocks will be identified first.

3. Associate a flag with each gene. These flags are initially all cleared, and when set, indicate that the gene has already been assigned to another subblock.

4. Repeat steps 5-7 through all sorted genes {gⱼ}.

5. If the flag for gⱼ is set, skip the gene.

6. Generate a subblock starting with gⱼ according to the procedure described below.

7. Convert the subblock to matrix form. If the submatrix is not invertible, go back and generate a different subblock, or put the gene at the end of the list and try again later. If the submatrix is invertible, a valid subblock has been identified. Therefore all genes belonging to the subblock are flagged.

In constructing a subblock, the starting gene is placed into the GeneList. For each new gene, gₐ (including the first one) added to the GeneList, the following actions are taken:

8. Select an oligonucleotide oₓ from Oligonucleotide Set(gₐ), preferably with the lowest possible degeneracy, that is not already in the Oligonucleotide List. Removal of oligonucleotides which are already present in another subblock, should be avoided unless a higher degeneracy of oligonucleotide was chosen.

9. Add oligonucleotide oₓ to the Oligonucleotide List 10. For each gene in GeneSet(oₓ), add the gene to the GeneList. If any of the genes has already been assigned to a subblock, then all genes in that subblock are entered into the GeneList, and all the oligonucleotides in the subblock are put into the OligonucleotideList.

The skilled artisan will readily appreciate that many of the steps recited supra will be optional and need not be performed in order to implement the algorithm of this invention.

Preferably, steps 8-10 are iteratively repeated for each gene added to the gene list so that an oligonucleotide probe is added to the Oligonucleotide List for each gene added to the Gene List, and so forth. In preferred embodiments, when the average degeneracy is at or close to one, this recursive procedure will usually terminate very quickly, and the subblocks are suitably small. Thus, in one preferred embodiment the algorithm is iteratively repeated for each subblock until, for each gene gₐ associated with the gene list for a particular subblock, all oligonucleotide probes oₓ which hybridize to the gene gₐ (and, optionally, have a Degen(oₓ) that is less than or equal to a selected threshold T) are assigned to the particular subblocks. In such embodiments, it is anticipated that there may be some genes g_{c} that hybridize only to probes having a high level of degeneracy so that MinDegen(g_{c}) is greater than the selected threshold T. Generally, such genes g_{c} are not considered when assigning genes and probes to subblocks according to the above algorithm.

In another preferred embodiment, the algorithm is iteratively repeated for each subblock until, for each oligonucleotide probe oₓ assigned to the particular subblock, all genes gₐ that hybridize to the oligonucleotide probe oₓ are associated with the gene list for the particular subblock.

These two preferred embodiments are not exclusive of one another. Thus, in still another preferred embodiment the algorithm may be iteratively repeated for each subblock until: (i) for each gene gₐ associated with the gene list for the subblock, all oligonucleotide probes oₓ hybridizing to the gene gₐ (and optionally having a Degen(oₓ) that is less than or equal to a selected threshold T) are assigned to the subblock; and (ii) for each oligonucleotide probe oₓ assigned to the particular subblock, all genes gₐ that hybridize to the oligonucleotide probe oₓ are associated with the gene list for the particular subblock.

In still other embodiments, the steps may be repeated for a set number of iterations, e.g., selected by a user. For example, in other embodiments the iterative steps of the algorithm may be repeated for less than 100, less than 50 or less than 20 iterations. In particularly preferred embodiments, the steps are repeated for not more than ten, not more than five, not more than four, not more than three or not more than two iterations. In particularly preferred embodiment only a single iteration of the steps is performed.

If the average degeneracy is higher, then the algorithm must be adapted during subblock building to control the subblock size. In Example 3, an analytical model is presented for predicting the average degeneracy for the design of the n-mer array parameters, such that the degeneracy is suitably small and the simple algorithm above will suffice.

### 6.3. Example 3

### Probabilistic Degeneracy Model

This Example presents an analytical model to predict the average degeneracy for a specified genome with a particular oligonucleotide length, n. This model predicts the suitable value for n which can accommodate genomes ranging in size from a yeast to a mouse. The model is further extended to incorporate additional parameters arising from some potentially useful modifications to the hybridization procedure, such as length truncation mentioned earlier. By analyzing degeneracies for real genomic sequence data, the model is validated and its various extensions bear a very close correlation between measured and predicted values. Finally, the model is used to estimate the parameters that are suitable or required to achieve low average degeneracy for the yeast and mouse genome, and to demonstrate that these predictions are accurate.

Basic Model.

In consideration of a single gene of length l it is assumed that the immobilized n-mers are sufficiently far from the surface of the DNA chip (which can be achieved by using long linker molecules), and they are not too densely packed. This reduces steric interference during hybridization [16] so that any existence of size n along the gene is a potential location for binding to an n-mer. By sliding a window of size n along the gene, it is easy to see that there are

b(l, n)=1−n+l

binding positions (“sites”) in the gene. Usually it is the case that l>>n and the quantity b(l, n)≈l. Note that we make the assumption that a tethered oligonucleotide never overhangs the strand with which it is binding, even if mismatches are allowed.

Since there are b binding sites and Nₒ different oligonucleotides, then the probability of any one particular oligonucleotide binding to a gene is given by

$$p ( l , n , m ) = \frac{b ( l , n )}{{N}_{o}} .$$

If a completely random distribution of bases in the genome has been assumed, randomness simply ensures that all oligonucleotides have equal probability of binding everywhere.

As shown earlier, the degeneracy, d(n, m), may be defined as the number of genes to which an oligonucleotide can hybridize, given a maximum number of allowed mismatches, m. In this model, d(n, m)=N_{g}p(l, n, m), and the average degeneracy over all genes in a particular can be easily computed.

$$\begin{matrix}λ ( n , m ) = ⟨ d ( n , m ) ⟩ = \frac{1}{{N}_{g}} {\sum }_{j = 1}^{{N}_{g}} {N}_{g} p ( {l}_{j} , n , m ) \\ = {\sum }_{j = 1}^{{N}_{g}} \frac{1 - n + l}{{N}_{o}} \\ = \frac{{N}_{g}}{{N}_{o}} ( 1 - n + \frac{1}{{N}_{g}} {\sum }_{j = 1}^{{N}_{g}} {l}_{j} ) \\ = \frac{{N}_{g}}{{N}_{o}} ( 1 - n + ⟨ l ⟩ )\end{matrix}$$

Where (l) is the average gene length for the given genome. This is essentially a Poisson distribution, and hence we have denoted the mean value by λ(n, m). (The mean value of a Poisson distribution with parameter value λ is equal to λ itself.) This can also be interpreted as a Binomial distribution, where the probability of “success” is p and the number of trials is N_{g}.

Basically a computer program gathers degeneracy histograms from real genomic data based on selected values for the parameters n and m, and gene truncation length. The program reads through all the sequences of a genome and counts how many different genes contain each of the 4ⁿ oligonucleotides as a subsequence (allowing for up to m mismatches), and writes these values to an output file.

In this way, degeneracy histograms have been generated from two public gene sequence sets: yeast (Saccharomyces cerevisiae) and mouse (Mus musclus). Although the mouse sequence data set is not a complete genome, it is sufficient for the present purpose. These two genomes were selected as representing two ends of a wide spectrum of genome size, and thus are helpful in identifying suitable values for n. Also, yeast and mouse are among the organisms most commonly used in genetics experiments, including expression analysis.

The yeast genome was downloaded from the Saccharomyces Genome Database at Stanford University. (http://genome-www.stanford.edu/Saccharomyces/. File :ftp://genome-ftp.stanford.edu/pub/yeast/yeast_ORFs/orfs_coding.fasta.Z). Only the coding regions of the genome were used because these are the parts which get transcribed into mRNA. For this sequence, parameter values were N_{g}=6306 and (l)≈1420.

Gene sequences for the mouse genome were downloaded from the UniGene system at the National Center for Biotechnology Information, NCBI. (http://www.ncbi.nlm.nih.gov/UniGene/. file ftp://ftp.ncbi.nlm.nih.gov/repository/UniGene/Mm.seq.uniq.Z, Build 74 was downloaded). Gene sequences in the UniGene system are grouped into clusters with similar sequences and the sequences in the file downloaded contain one representative sequence from each cluster. The sequences consist of known genes (which are transcribed into RNA) and expressed sequence tags (ESTs) which have been discovered in cDNA libraries). The parameter values for this data set are N_{g}=75963 and (l)≈471.

For the yeast genome, degeneracy measurements were carried out for n-values ranging from 7 to 12; for the set of mouse genes, n-values ranged from 9 to 14. m-values of 0 and 1 were used in both cases.

Although the Poisson model does not accurately predict the exact shapes of the simulated degeneracy histograms, the mean (expected) values of λ correspond very well between the model and the data. For the case of no mismatches (m=0), the results are listed in Table 1. When the mean value is large, the Poisson distribution tends to be narrowly distributed around the mean, whereas the computed histogram distribution is wider and is strongly asymmetric, with a sharp rise at low degeneracy values. If the Poisson distribution is convolved as a function of gene length l with the actual length distribution in the genome, most of the width seen in the actual degeneracy histograms can be recovered. Further improvements are obtained by convolving with the distribution of n-mers in the genome (which has been assumed to be uniform so far).

TABLE 1
Average degeneracy with 0 mismatches.

| organism | n-mer size | λ¹ (actual) | λ (theory) |
|---|---|---|---|
| yeast | 7 | 479.3 | 544.2 |
| yeast | 8 | 130.2 | 135.9 |
| yeast | 9 | 33.42 | 33.96 |
| yeast | 10 | 8.420 | 8.485 |
| yeast | 11 | 2.110 | 2.120 |
| yeast | 12 | 0.5275 | 0.5295 |
| mouse | 9 | 130.2 | 134.1 |
| mouse | 10 | 32.66 | 33.44 |
| mouse | 11 | 8.161 | 8.343 |
| mouse | 12 | 2.037 | 2.081 |
| mouse | 13 | 0.518 | 0.519 |
| mouse | 14 | 0.127 | 0.130 |

¹Measurements of λ (the average degeneracy) from the yeast and mouse genomes are compared with predictions from the analytical model.

The analytical model consistently overestimates the value of λ, with a greater discrepancy as λ increases (corresponding to smaller values of n). This effect is understood as due to clipping errors. For any oligonucleotide, the maximum degeneracy is N_{g}, i.e., the total number of genes. Under conditions where the analytical model predicts a value of λ which is close to the maximum degeneracy, the histogram obtained from the data is highly “clipped”. Thus, because the histogram is lacking the higher degeneracy values, the computed average value is necessarily lower than the prediction. Since the model is directed to cases where λ=1, “clipping effects” are not considered to be a problem, and this Example does not model the histograms to reduce “clipping effects”.

As a result of overestimation of empirical values, any constraints placed on parameters to ensure that the average degeneracy is below a certain threshold should be more stringent than necessary. Therefore the result will be a conservative prediction of the tractability of the algorithm.

Mismatch Model.

Mismatches can be handled in a rather simple manner. The occurance of mismatches in duplexes between immobilized oligonucleotides and genes increases the probability, p(l, m, n), of binding.

For m=0, there is only one resulting n-mer sequence which is fully complementary to a given n-mer sequence. When m=1, there are 3n+1 such complementary sequences which include the possibility of a perfect match. (For the mismatches, one of the n positions is switched to one of the three other bases). In the general case, c(m) complementary sequences will occur when m mismatches are permitted, where c(m) may be provided by the relation:

$$c ( m ) = {\sum }_{k = 0}^{m} ( \begin{matrix}n \\ k\end{matrix} ) {3}^{k} = {\sum }_{k = 0}^{m} \frac{n !}{k ! ( n - k ) !} {3}^{k}$$

Thus the probability of binding is expected to increase by this factor, so that the average degeneracy may be provided by the relation:

$$⟨ d ( n ) ⟩ = \frac{{N}_{g}}{{N}_{0}} ( 1 - n + ⟨ L ⟩ ) \times c$$

where c may be provided by the formula for c(m) given above.

An equivalent formulation is that the total number of oligonucleotides is effectively reduced by a factor of c(m), such that

$${N}_{o} , eff = \frac{{4}^{n}}{c ( m )}$$

Thus all the formulae described in the model above should still be valid if Nₒ is replaced everywhere with N_{o,eff}. In a sense, the size of the n-mers has been decreased: a larger array size (n) is required in order to achieve the same average degeneracy as a case with smaller m.

These results of the model with m=1 are compared with actual measurements in Table 2. The data is derived from the same genome database as above. As for the perfectly matched case, the correspondence here between prediction and measurement is excellent.

TABLE 2
Average degeneracy with 1 mismatch.

| organism | n-mer size | λ² (actual) | λ (theory) |
|---|---|---|---|
| yeast | 7 | 4190 | 11970 |
| yeast | 8 | 2120 | 3399 |
| yeast | 9 | 790.0 | 950.9 |
| yeast | 10 | 2.45.8 | 263.0 |
| yeast | 11 | 70.29 | 72.07 |
| yeast | 12 | 19.39 | 19.59 |
| mouse | 9 | 3308 | 3754 |
| mouse | 10 | 976.2 | 1037 |
| mouse | 11 | 273.8 | 283.6 |
| mouse | 12 | 74.96 | 77.00 |
| mouse | 13 | 20.27 | 20.77 |
| mouse | 14 | 5.442 | 5.569 |

²Comparison of λ as measured from the yeast and mouse genome with the predictions of the analytical model.

It is noted that the methods of the invention are not limited to the particular mismatch model described above and that other models, which will be readily apparent to the skilled artisan, may also be used. For exdample, a variety of thermodynamic models for nucleic hybridization are well known in the art [1, 6, 8, 14, 18]. Using such models, a skilled artisan may readily determine (e.g, by calculation) a number of sequences c(n) of length n that will hybridize or are capable of hybridizing to an oligonucleotide probe of length n. Thus, for a given collection of No different oligonucleotide probes having a particular sequence length n (for example, a collection of N₀=4ⁿ probes on a universal array) the number of sequences <c(n)> that may hybridize, on average, to a given probe can be readily calculated or otherwise determined. The probability of binding is expected to increase by this factor so that the average probe degeneracy may be provided by the relation

$$⟨ d ( n ) ⟩ = \frac{{N}_{g}}{{N}_{0}} ( 1 - n + ⟨ L ⟩ ) \times ⟨ c ( n ) ⟩$$

Extensions to the Parameter Space.

As described in Example 2, the average degeneracy must have a value close to one (unity) in order that the matrix inversion of Equation (1) is tractable. We have previously discussed the possibility of truncating mRNA transcripts to effectively reduce the sequence space of the genome. Here we extend our analytical model to handle this possibility and again compare its predictions with measurements from real sequence data.

The two different approaches to truncation can easily be incorporated into the model. In order to model the effect of a decrease in length of all transcripts by an amount (ΔL), (l) is replaced with the average gene length, (l)-(ΔL). To model the result of truncating to a small fixed length, we need only change quantity (l) to L.

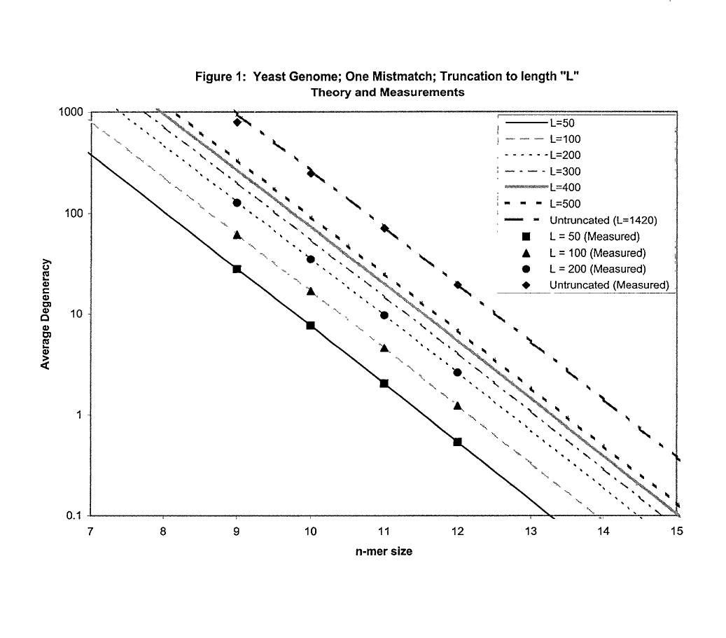

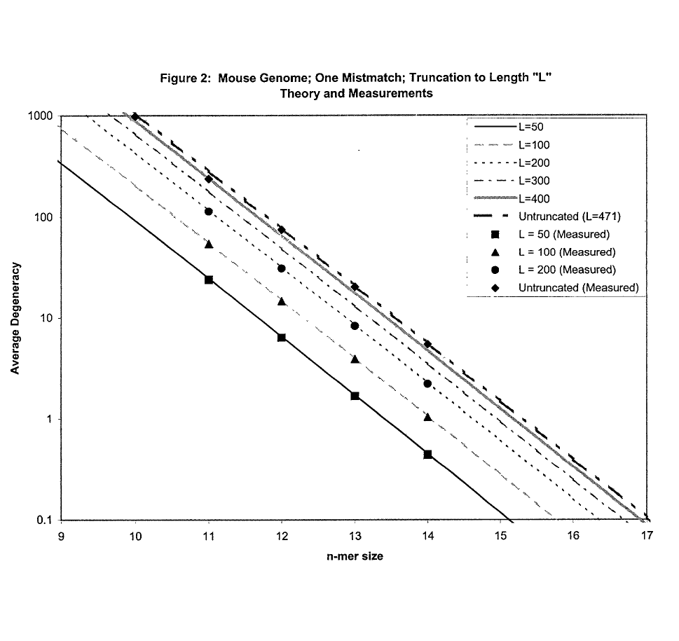

FIGS. 1 and 2 compare average degeneracies computed from the raw data set with predictions of the analytical model for yeast and mouse, respectively. In our computations, we assumed a truncation to length L=50, 100, and 200 from the 5′-end of the mRNA, and assumed that single mismatches were possible. Theoretical lines were also included for L=300 and 400 as a helpful tools when designing the n-mer array parameters. As for previous cases, the measured and theoretical values are extremely close. It is interesting that the assumption of a random distribution of bases throughout the genome continues to hold in spite of the reduction in sequence space resulting from truncation.

Predictions.

There is good correlation between actual and predicted average degeneracies over a range of values for the parameters n and L as shown in FIGS. 1 and 2. This indicates that the formulae presented earlier can be used for making accurate predictions. FIGS. 1 and 2 illustrate the comparison of λ as measured from the yeast and mouse genome with the predictions of the analytical model. The solid lines are plots of the equation for λ given in the text with appropriate modifications for length truncation. The markers represent the measured values for certain values of n-mer size n and truncation length L, determined by counting occurrences of subsequences in the genome sequences.

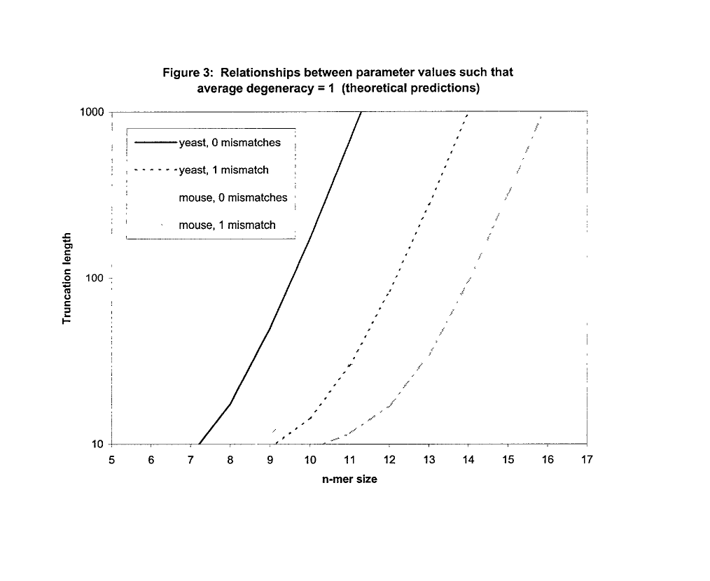

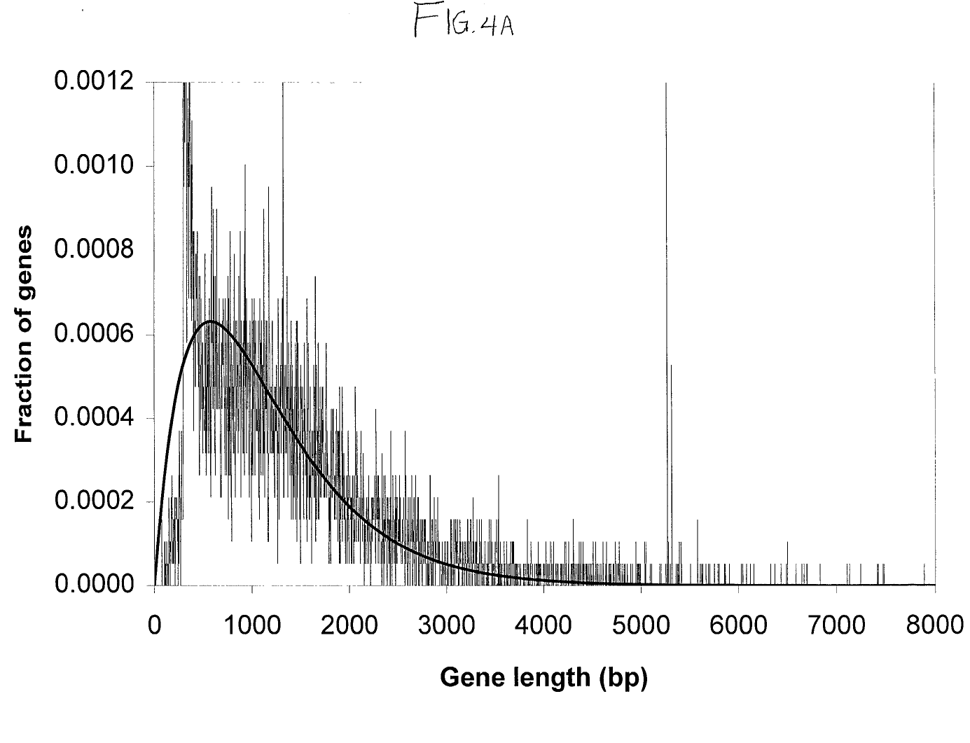

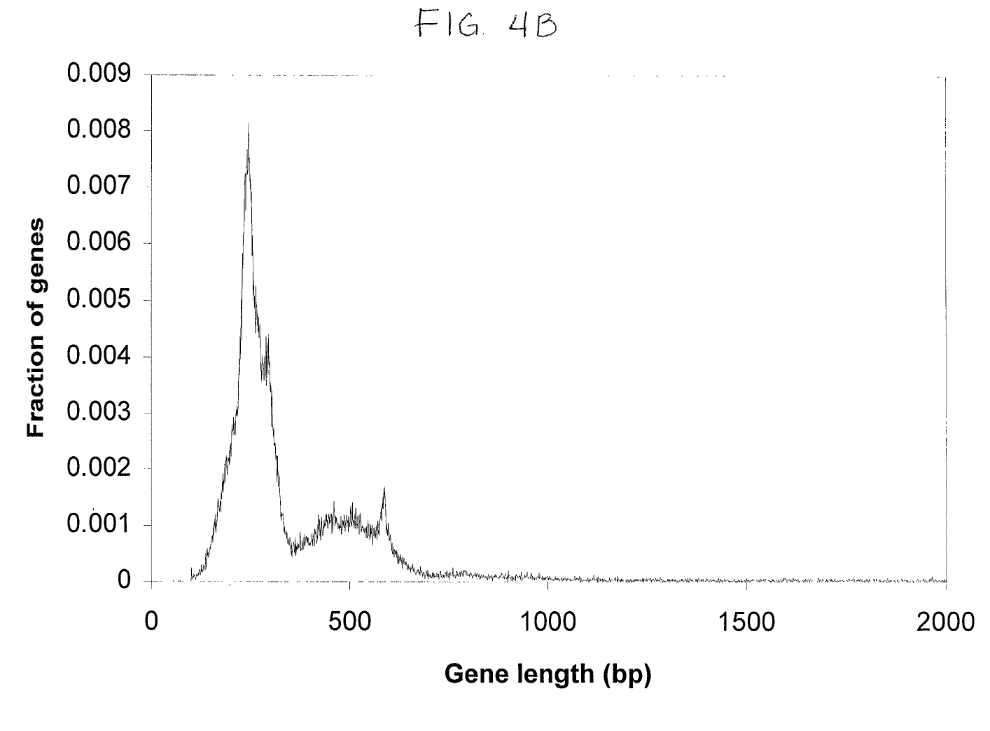

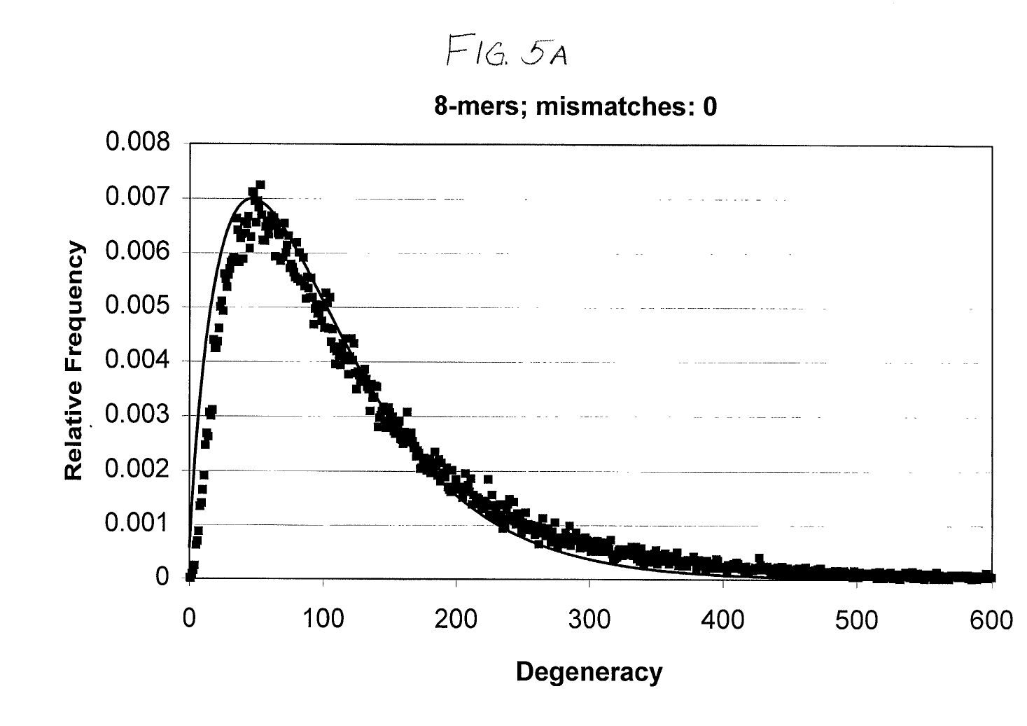

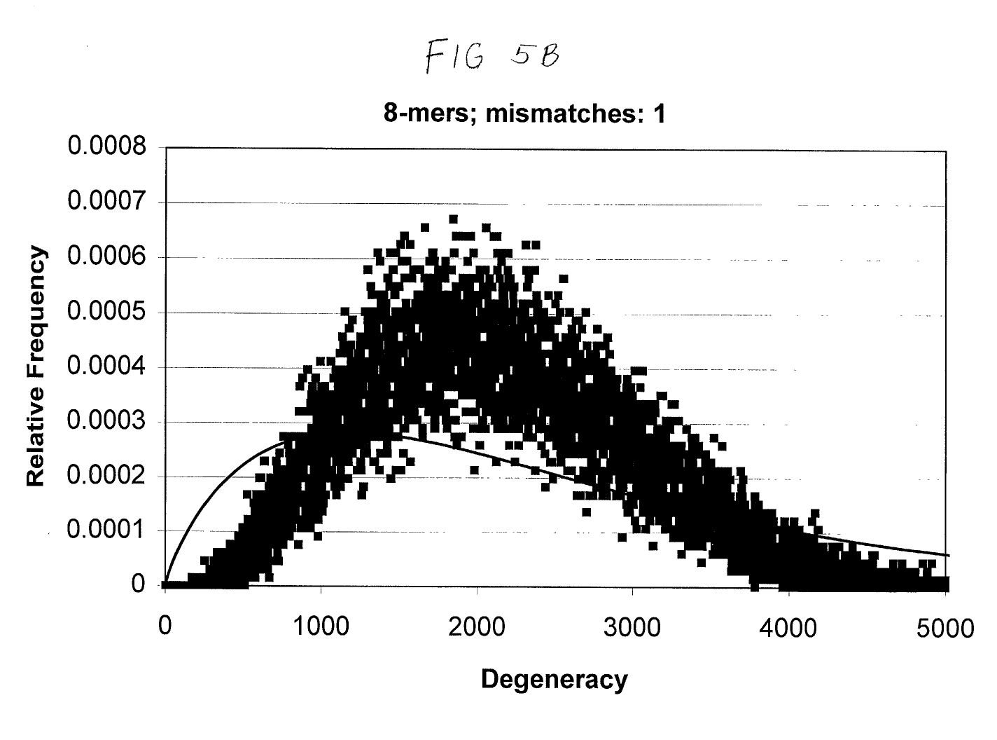

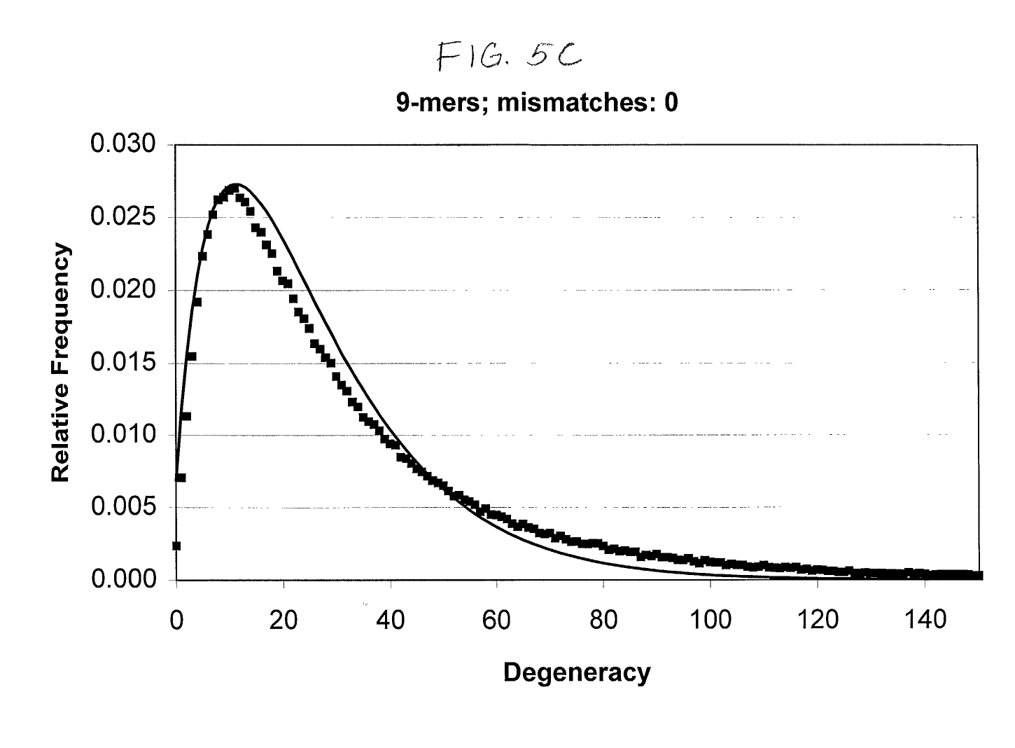

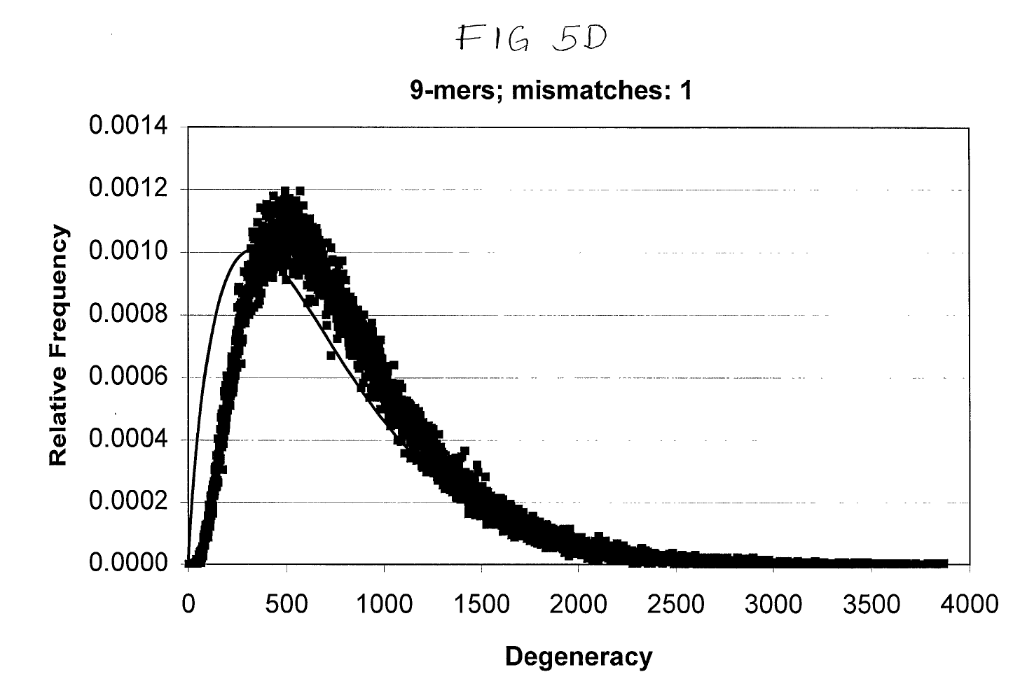

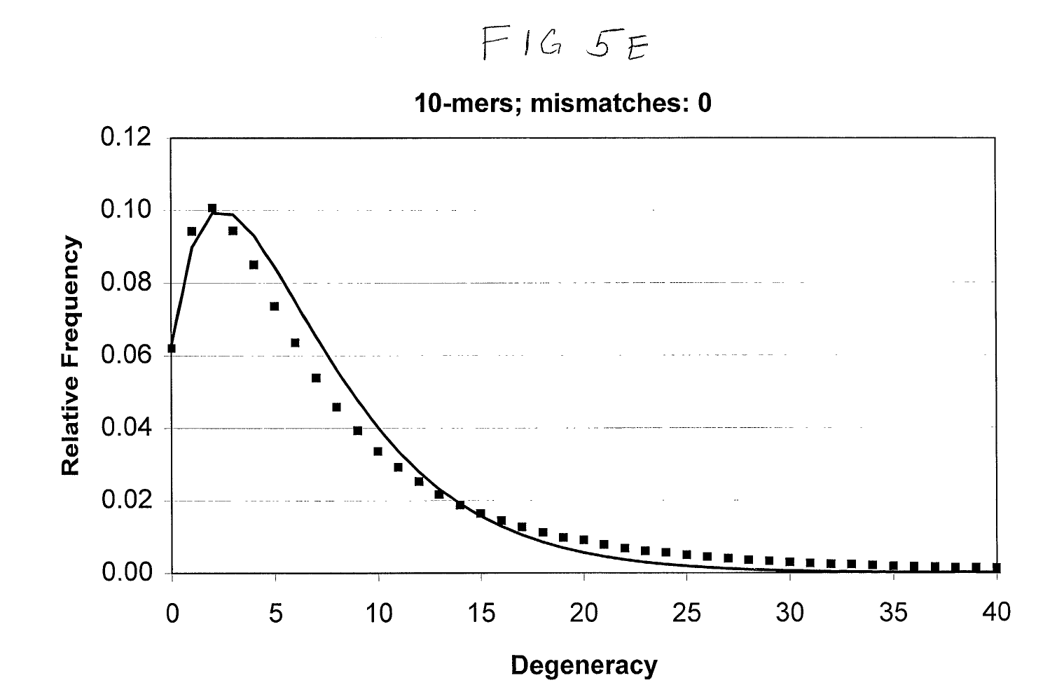

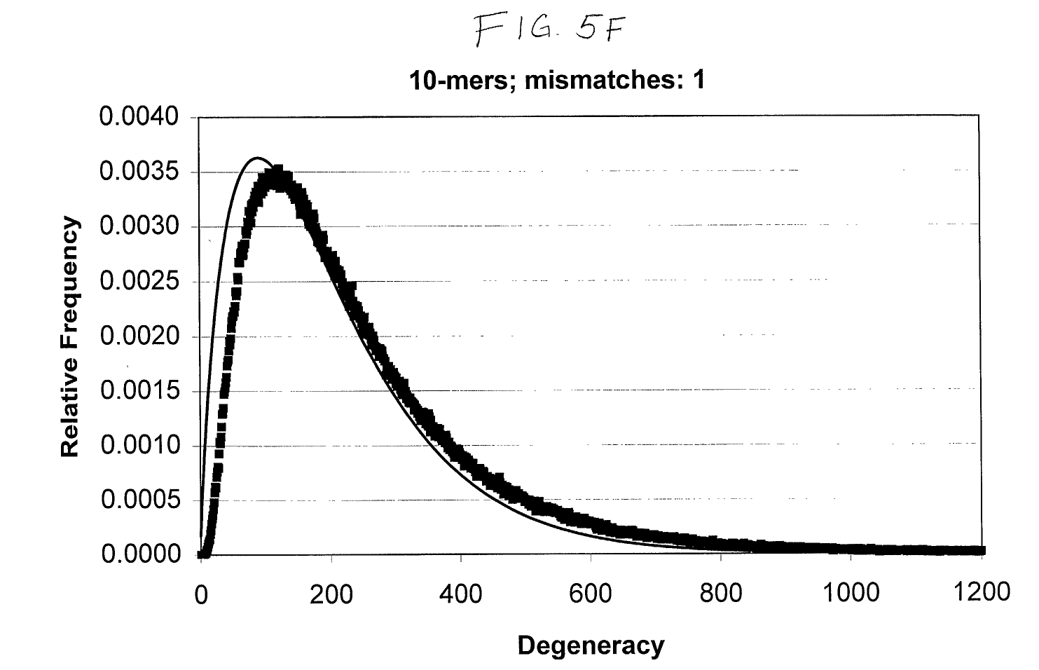

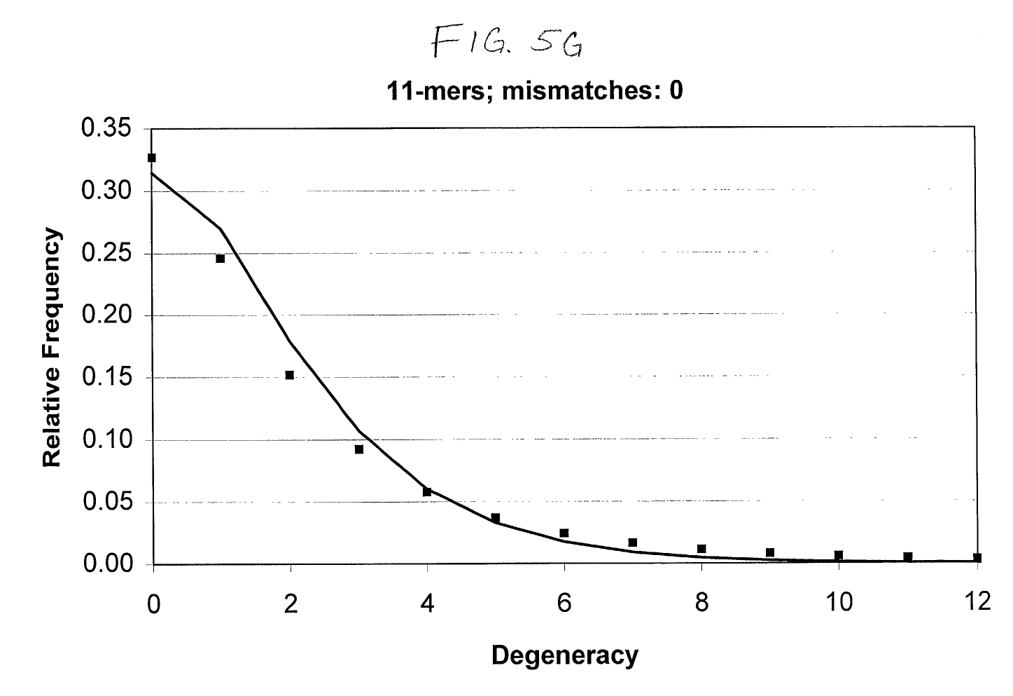

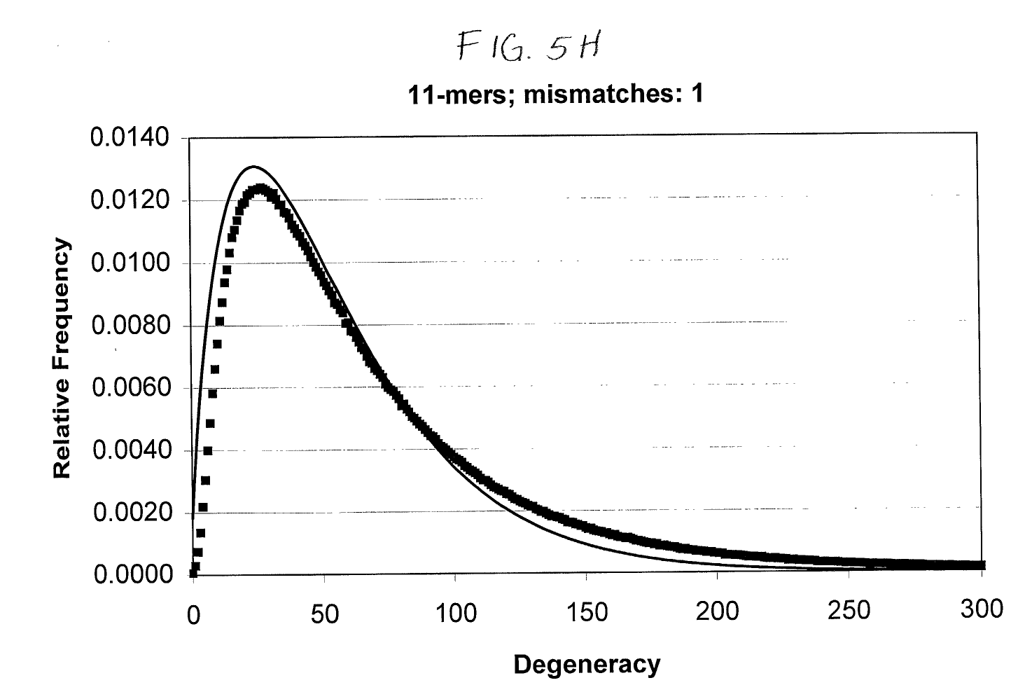

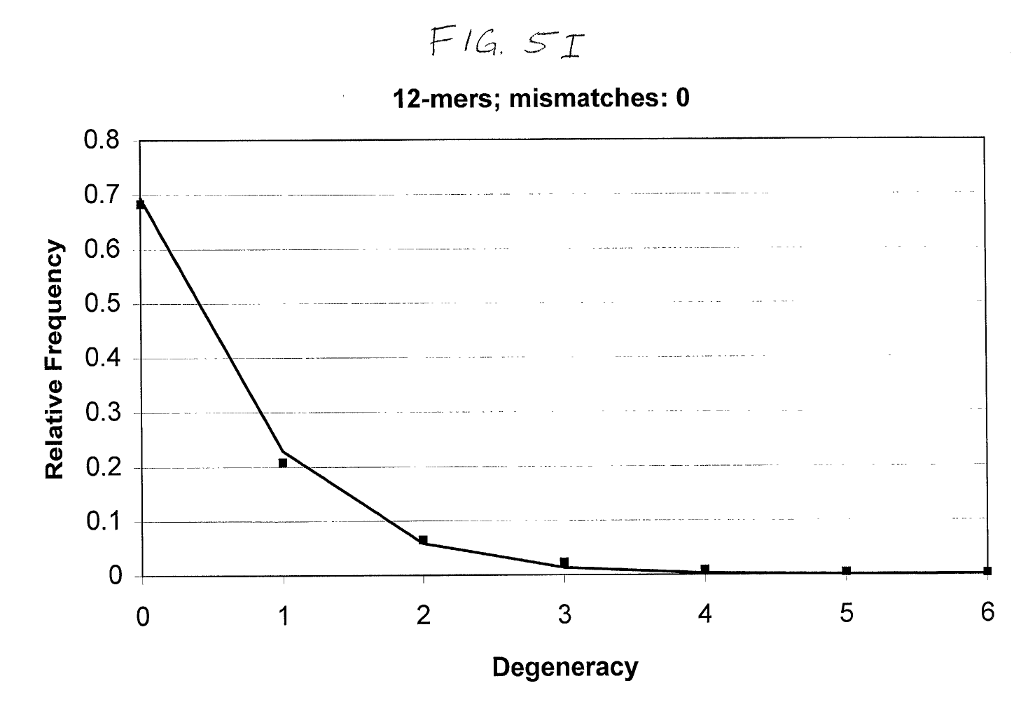

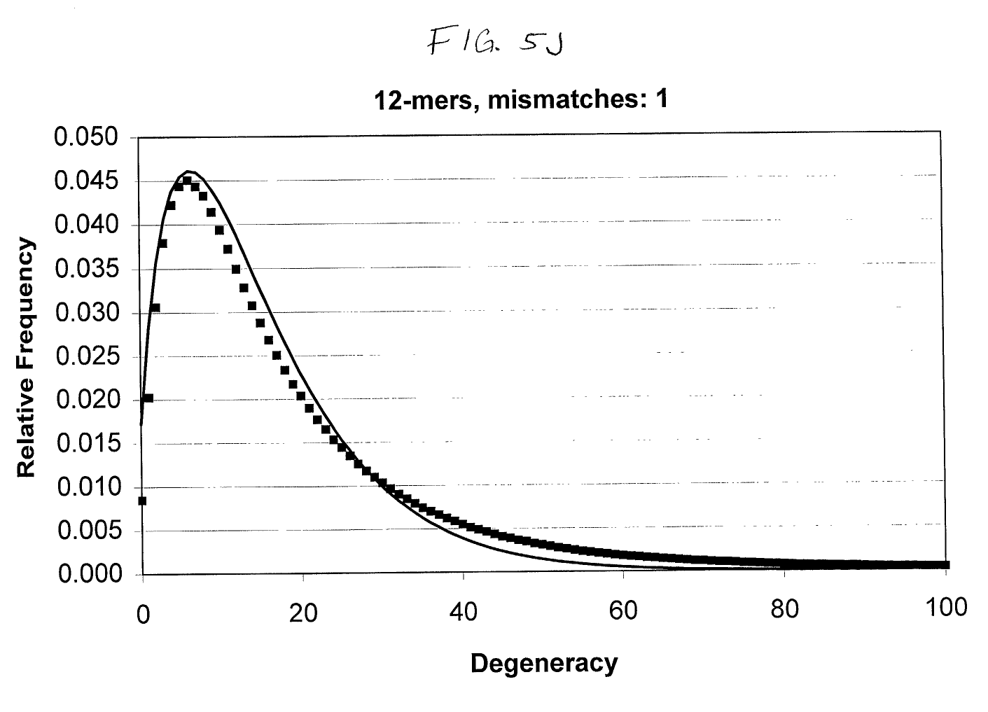

FIG. 3 illustrates the relationship between n-mer size and truncation length such that the average degeneracy, λ is unity. Theoretical curves for both mouse and yeast and shown, for the two cases, no mismatches, and one mismatch allowed. FIG. 3 has the same theoretical predictions in a different format, each line represents the relationship between the parameter n and truncation length required in order to achieve a target average degeneracy of unity (i.e. which is important so that the algorithm is tractable).

These Figures can be used to predict the parameter values. Assuming that a single base mismatch is allowed for the mouse genome, we can see that the target degeneracy is nearly achieved with a truncation length to 50 oligonucleotides and n-mers of length 13. If n=15 could be achieved, then almost no truncation is required. Similarly, for the yeast genome, the target degeneracy is achieved with the truncation length is 50 and the n-mer size is 11. The average gene length in the yeast genome is larger than mouse, therefore there is a jump up to n=14 in order to achieve the target degeneracy without truncation.

The results so far consider the average degeneracy of all n-mers on a universal array. However, when degeneracy is sufficiently low only a small subset of those oligonucleotides is required to monitor individual gene expression levels. A logical starting point is to consider, for each gene, the minimum degeneracy n-mer to which it can bind. Transcripts gᵢ for MinDegen(gᵢ) is equal to one are obvious trivial cases; i.e., expression levels of these transcripts may be readily solved merely by measuring the hybridization signal of this minimum degeneracy oligonucleotide. Of the remaining transcripts in a genome (e.g., in a collection of nucleic acids), those which share their minimum degeneracy oligonucleotide only with other transcripts gᵢ for which MinDegen(gᵢ)=1 are also trivial. Expression levels for these genes may be determined after subtracting the hybridization contribution from the other transcripts (which, in turn, is trivially determined from the hybridization level of their respective minimum degeneracy oligonucleotides).

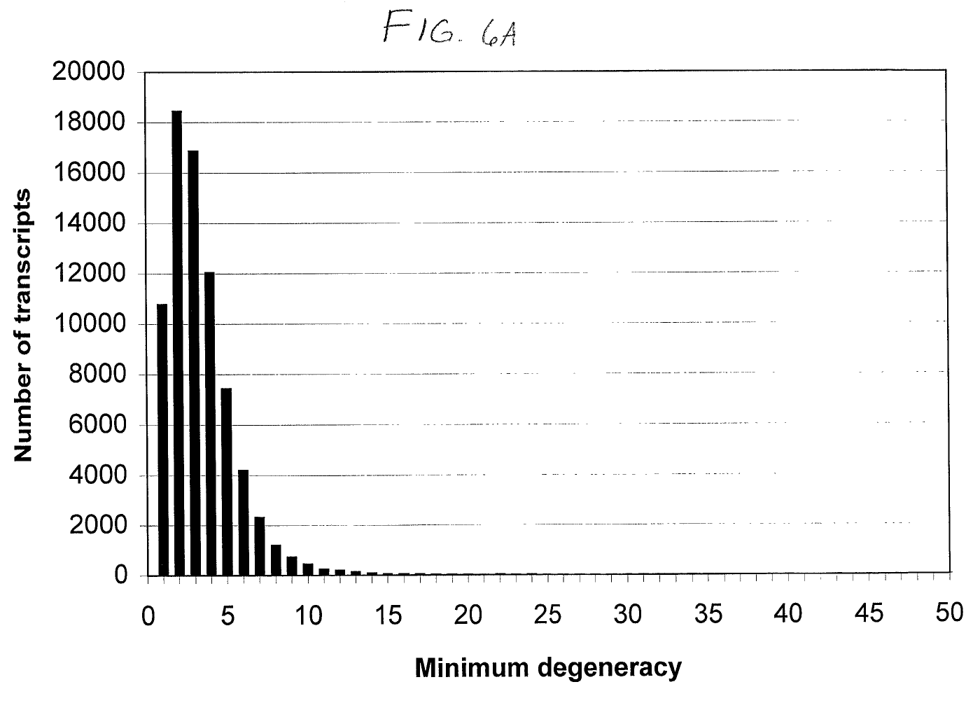

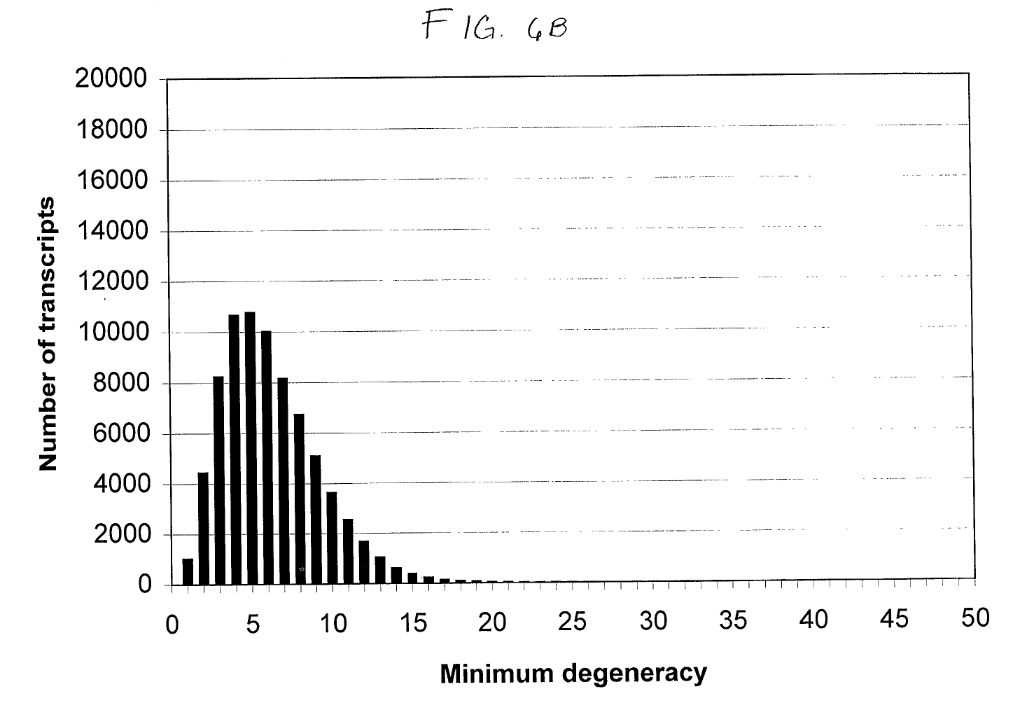

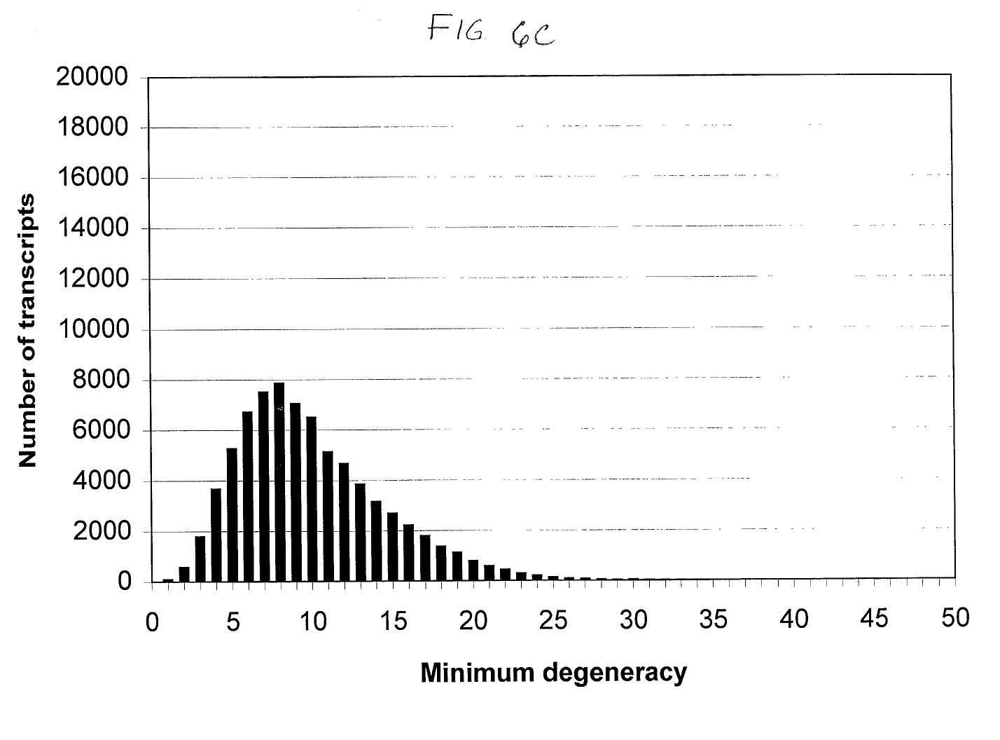

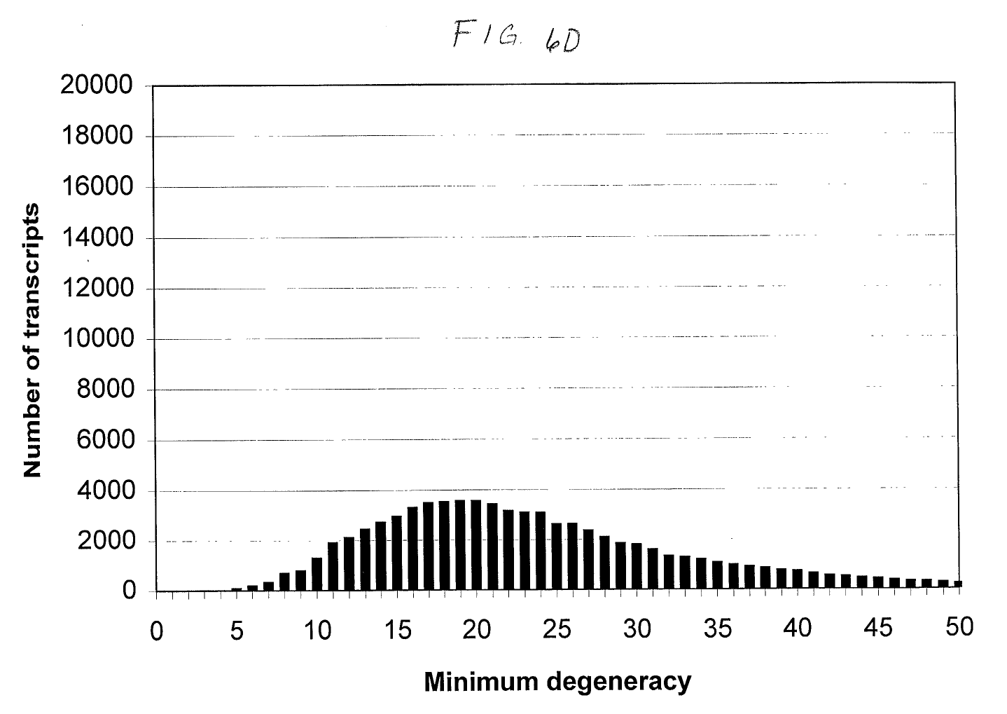

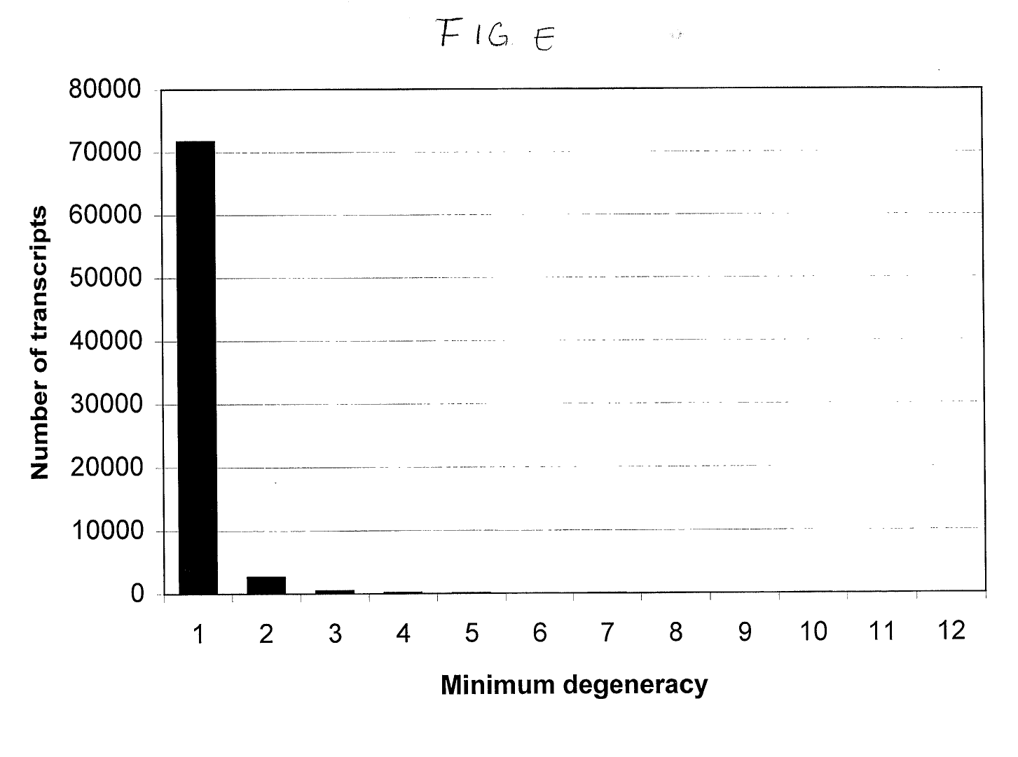

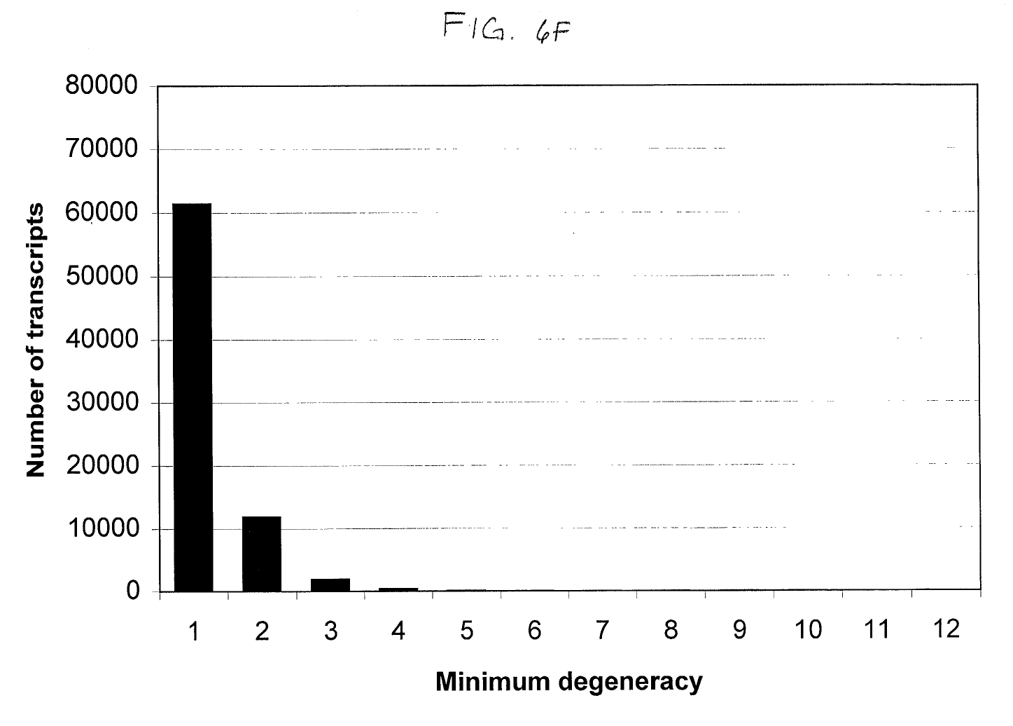

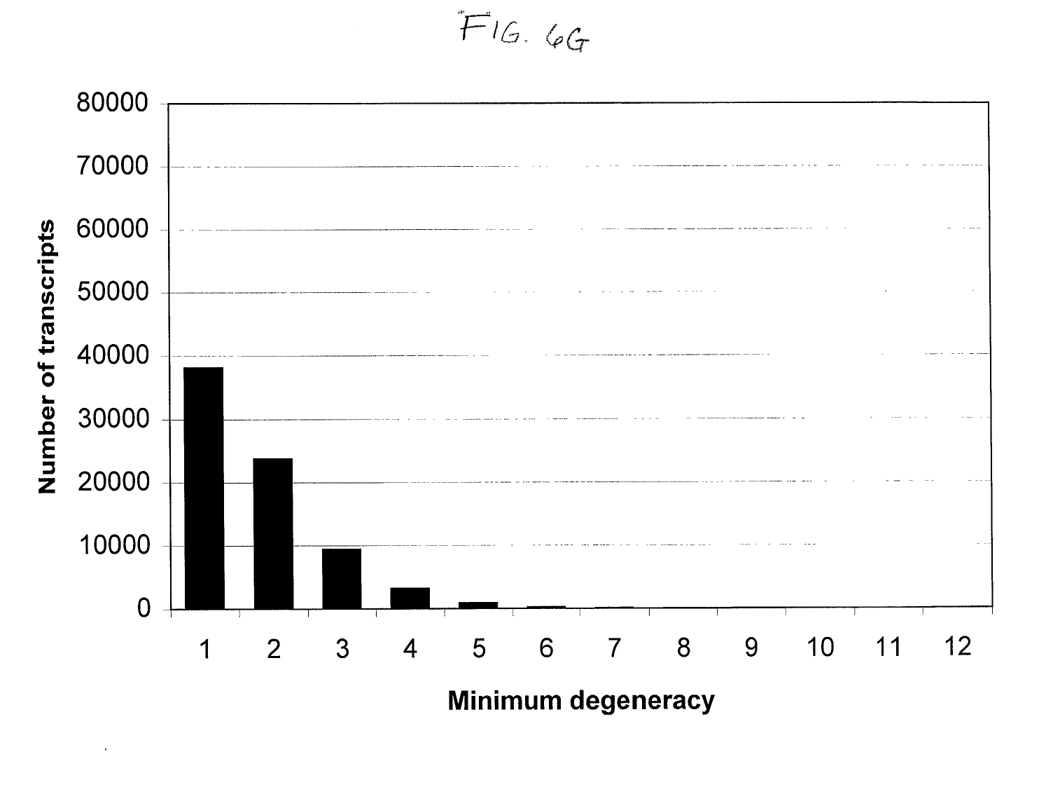

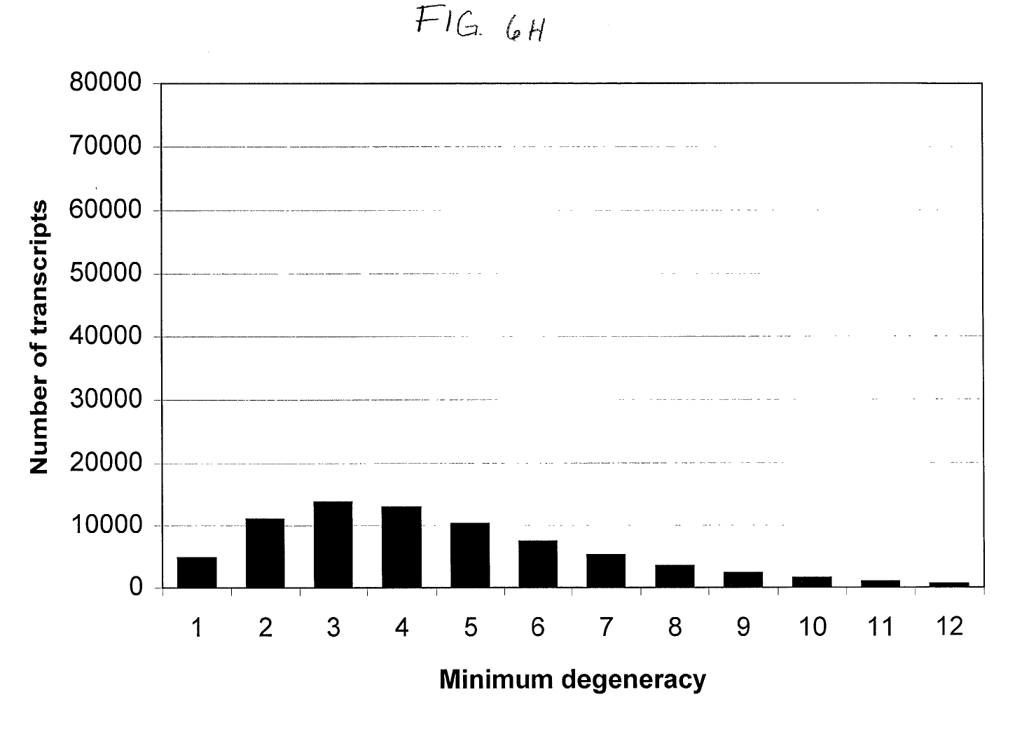

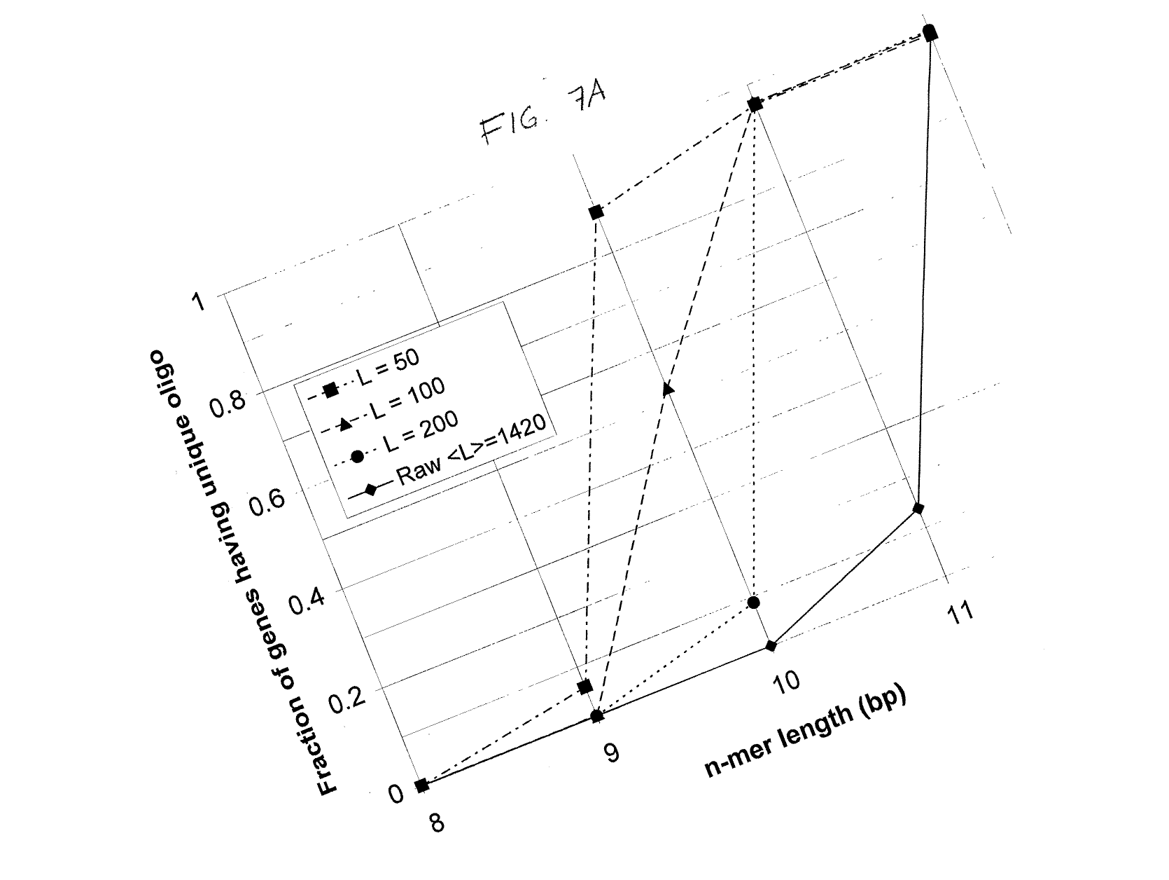

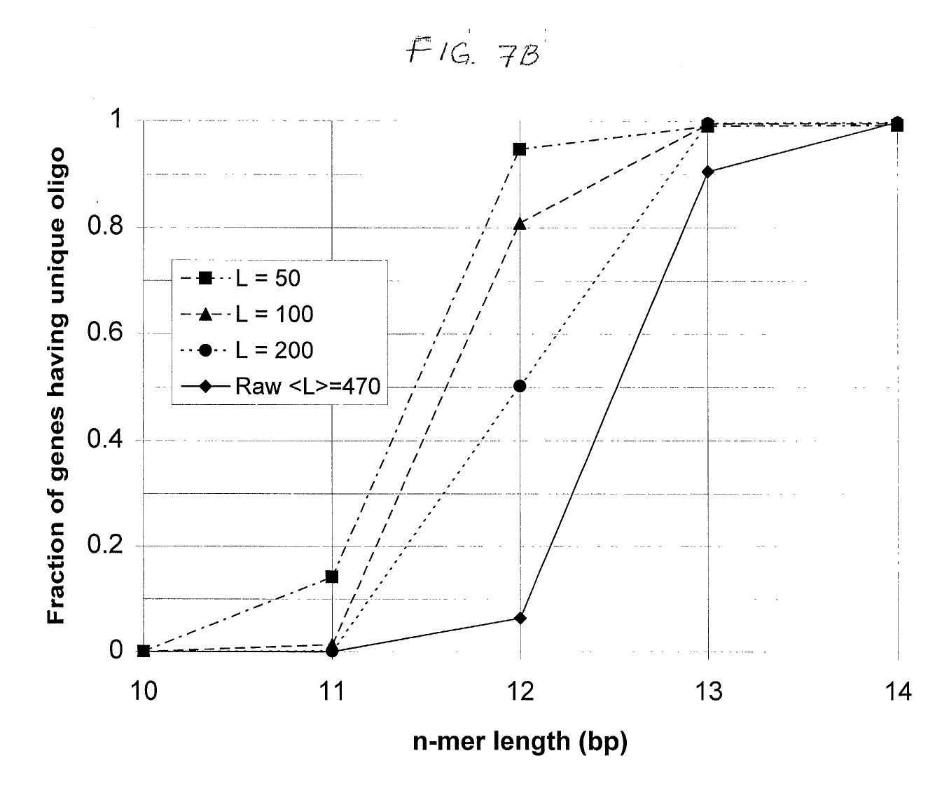

Assuming the lowest degeneracy of oligonucleotide is chosen from each gene, modified degeneracy histograms were computed for various values of the parameters n and L (see, FIGS. 6A-H). For yeast (FIG. 7A) with a 10-mer array (i.e., n=10) and a truncation length L of 50 bases, nearly 90% of the transcripts have a minimum degeneracy of 1, corresponding to an average degeneracy of ≈1. The data indicated that expression levels for most transcripts in yeast (about 98%) can be readily solved given these parameter values. Most of the subblocks in the matrix H′ will have a size 1×1 and so the matrix inversion will be trivial. It is further noted that the value n=10 is one base less than what was predicted using only the analytical model.

For mouse (FIG. 7B) it was found that a truncation to a length of 50 or 100 and an array of n=12 results in 80% or 90%, respectively, of genes with a degeneracy of 1.

These experiments indicate that universal n-mer arrays with probe lengths between about 10-15 bases are useful as tools for studying gene expression. Other applications of n-mer arrays include DNA sequencing by hybridization, the study of DNA binding proteins, and genomic fingerprinting. Some of the most significant advantages of these n-mer arrays are that: 1) they are universal, so that the same chip can be used to study any organism, and 2) the data can be reanalyzed as more genomic sequence data is accumulated (rather than performing another experiment).

It will be appreciated by persons of ordinary skill in the art that the examples and preferred embodiments herein are illustrative, and that the invention may be practiced in a variety of embodiments which share the same inventive concept.

### 7. BIBLIOGRAPHY

[1] K. J. Breslauer, R. Frank, H. Blöcker, and L. A. Marky. Proc. Natl. Acad. Sci. USA, 83:3746-3750, 1986.

[2] M. L. Bulyk, E. Gentalen, D. J. Lockhart, and G. M. Church. Quantifying dna-protein interactions by double-stranded dna arrays. Nature Biotechnology, 17:573-577, 1999.

[3] M. Chee, R. Yang, E. Hubbell, A. Bemo, X. C. Huang, D. Stem, J. Winkler, D. J. Lockhart, M. S. Morris, and S. A. Fodor. Accessing genetic information with high-density dna arrays. Science, 274:610-614, 1996.

[4] Peter B. Dervan and Roland W. Bürli. Sequence-specific dna recognition by polyamides. Current Opinion in Chemical Biology, 3:688-693, 1999.

[5] S. Drmanac, D. Kita, I. Labat, B. Hauser, J. Burczak, and R. Dramanac. Accurate sequencing by hybridization for dna diagnostics and individual genomics. Nature Biotechnology, 16:54-58, 1998.

[6] Alexander V. Fotin, Aleksei L. Drobyshev, Dmitri Y. Proudnikov, Alexander N. Perov, and Andrei D. Mirzabekov. Parallel thermodynamic analysis of duplexes on oligodeoxyribonucleotide microchips. Nucleic Acids Research, 26:1515-1521, 1998.

[7] Zhen Guo, Qinghua Liu, and Lloyd M. Smith. Enhanced discrimination of single nucleotide polymorphisms by artificial mismatch hybridization. Nature Biotechnology, 15:331-335, April 1997.

[8] Jörg D. Hoheisel. Sequence-independent and linear variation of oligonucleotide DNA binding stabilities. Nucleic Acids Research, 24(3):430-432, 1996.

[9] Gabor L. Igloi. Variability in the stability of dna-peptide nucleic acid (pna) single-base mismatched duplexes: Real-time hybridization during affinity electrophoresis in PNA-containing gels. Proc. Natl. Acad. Sci. USA, 95:8562-8567, July 1998.

[10] S. O. Kelley, E. M. Boon, J. K. Barton, N. M. Jackson, and M. G. Hill. Single-base mismatch detection based on charge transduction through DNA. Nucleic Acis Research, 27(24):4830-4837, Dec. 15, 1999.

[11] I. V. Kutyavin, I. A. Afonina, A. Mills, V. V. Gorn, E. A. Lukhtanov, E. S. Belousov, M. J. Singer, D. K. Walburger, S. G. Lokhov, A. A. Gall, R. Dempcy, M. W. Reed, R. B. Meyer, and J. Hedgpeth. 3′-minor groove binder-DNA probes increase sequence specificity at PCR extension temperatures. Nucleic Acis Research, 28(2):655-661, Jan. 15, 2000.

[12] Rogelio Maldonado-Rodriquez, Mercedes Espinosa-Lara, Pedro Loyola-Abitia, Wanda G. Beattie, and Kenneth L. Beattie. Mutation detection by stacking hybridization on genosensor arrays. Molecular Biotechnology, 11:13-25, 1999.

[13] J. Marton, Matthew, J. L. DeRisi, Holly A. Bennett, V. R. Iyer, Michael R. Meyer, Christopher J. Roberts, Rolan Stoughton, Julja Burchard, David Slade, Hongyue Dai, Douglas E. Bassett Jr., Leland H. Hartwell, P. O. Brown, and Stephen H. Friend. Drug target validation and identification of secondary drug target effects using DNA microarrays. Nature Medicine, 4:1293-1301, 1998.

[14] Björn Persson, Karin Stenhag, Peter Nilsson, Anita Larsson, Matthias Uhlen, and Per-A ke Nygren. Analysis of oligonucleotide probe affinities using surface plasmon resonance: A means for mutational scanning. Analytic Biochemistry, 246:34-44, 1997.

[15] M. Schena, D. Shalon, R. W. Davis, and P. O. Brown. Quantitative monitoring of gene expression patterns with a complementary DNA microarray. Science, 20:467-470, October 1995.

[16] M. S. Shchepinov, S. C. Case-Green, and E. M. Southern. Steric factors influencing hybridisation of nucleic acids to oligonucleotide arrays. Nucleic Acis Research, 25(6):1155-1161, 1997.

[17] Ronald G. Sosnowski, Eugene Tu, William F. Butler, James P. O'Connell, and Michael J. Heller. Rapid determination of single base mismatch mutations in DNA hybrids by direct electric field control. Proc. Natl. Acad. Sci. USA, 94:1119-1123, February 1997.

[18] E. M. Southern, U. Maskos, and J. K. Elder. Analyzing and comparing nucleic acid sequences by hybridization to arrays of oligonucleotides: Evaluation using experimental models. Genomics, 13:1008-1017, 1992.

[19] T. Spellman, Paul, Gavin Sherlock, Michael Q. Zhang, Vishwanath R. Iyer, Kirk Anders, Michael B. Eisen, Patrick O. Brown, David Botstein, and Bruce Futcher. Comprehensive identification of cell cycle-regulated genes of yeast Saccharomyces cerevisiae by microarray hybridization. Molecular Biology of the Cell, 9:3273-3297, December 1998.

[20] Andrey A. Stomakhin, Vadim A. Vasilisko, Edward Timofeev, Dennis Schulga, Richard Cotter, and Andrei D. Mirzabekov. DNA sequence analysis by hybridization with oligonucleotide microchips: Maldi mass spectrometry identification of 5mers contiguously stacked to microchip oligonucleotides. Nucleic Acids Research, 28(5):1193-1198, 2000.

[21] T. J. Yang, G. A. Lessard, and S. R. Quake. An apertureless near-field microscope for fluorescence imaging. Applied Physics Letters, 76:378-380, 2000.

[22] Gennady yershov, Victor Barsky, Alexander Belgovskiy, Eugene Kirillov, Edward Kreindlin, Igor Ivanov, Sergei Parinov, Dmitri Guschin, Aleksei Drobishev, Svetlana Dubiley, and Andrei Mirzabekov. DNA analysis and diagnostics on oligonucleotide microchips. Proc. Natl. Acad. Sci. USA, 93:4913-4918, May 1996.

[23] U.S. Pat. No. 5,922,591

[24] U.S. Pat. No. 5,143,854

[25] Fodor et al., Science, 251: 767-777 (1991)

[26] International Patent Publication No. WO 99/36760

[27] U.S. Pat. No. 5,525,464.

[28] U.S. Pat. No. 5,807,522

What is claimed is:

1. A method for analyzing data from hybridization of a sample to an array of oligonucleotide probes,
wherein the sample comprises a plurality of nucleotide sequences, each nucleotide sequence corresponding to a particular gene,
wherein some or all of the oligonucleotide probes are assigned to invertible subblocks such that each gene which hybridizes to an oligonucleotide probe assigned to a particular subblock does not hybridize to the oligonucleotide probes in the other subblocks, and
which method comprises a step of separately analyzing the data for the oligonucleotide probes in each subblock.

2. A method according to claim 1, wherein the array comprises a plurality (N₀) of oligonucleotide probes having a particular sequence length n so that all nucleic acid sequences having the particular sequence length are present on the array.

3. A method according to claim 2 wherein the particular sequence length n is from about 6 to about 20.

4. A method according to claim 3 wherein the particular sequence length n is from about 9 to about 16.

5. A method according to claim 4 wherein the particular sequence length n is from about 10 to about 12.

6. A method according to claim 4 wherein the particular sequence length n is from about 12 to about 15.

7. A method according to claim 1 wherein oligonucleotide probes are assigned to subblocks according to a method which comprises, for each subblock, steps of:
(a) associating a gene gₐ with a gene list for a subblock, wherein the gene gₐ is not already associated with a gene list for a subblock; and
(b) assigning an oligonucleotide probe oₓ to the subblock, wherein the oligonucleotide probe oₓ hybridizes to the gene gₐ,
wherein the steps are repeated for each subblock until each gene is associated with a gene list for a subblock.

8. A method according to claim 7, further comprising steps of:
(c) for each probe oₓ assigned to the subblock, associating genes g_{b} with the gene list for the subblock, wherein each gene g_{b} hybridizes to the probe oₓ; and
(d) for each gene g_{b} associated with the gene list, assigning an oligonucleotide probe o_{y} to the subblock, wherein the oligonucleotide probe o_{y} hybridizes to the gene g_{b}.

9. A method according to claim 8 wherein the steps of:
(c) associating genes g_{b} with the gene list for the subblock; and
(d) assigning an oligonucleotide probe o_{y} for each gene g_{b} associated with the gene list
are iteratively repeated for each oligonucleotide probe o_{y} assigned in step (d).

10. A method according to claim 9 wherein the steps (c)-(d) are repeated for not more than 100 iterations.

11. A method according to claim 10 wherein the steps (c)-(d) are repeated for not more than 50 iterations.

12. A method according to claim 11 wherein the steps (c)-(d) are repeated for not more than 20 iterations.

13. A method according to claim 12 wherein the steps (c)-(d) are repeated for not more than 15 iterations.

14. A method according to claim 13 wherein the steps (c)-(d) are repeated for not more than ten iterations.

15. A method according to claim 14 wherein the steps (c)-(d) are repeated for not more than five iterations.

16. A method according to claim 15 wherein the steps (c)-(d) are repeated for not more than four iterations.

17. A method according to claim 16 wherein the steps (c)-(d) are repeated for not more than three iterations.

18. A method according to claim 17 wherein the steps (c)-(d) are repeated for not more than two iterations.

19. A method according to claim 9 wherein the steps (c)-(d) are iteratively repeated until, for each oligonucleotide probe oₓ assigned to the particular subblock, all genes gₐ that hybridize to the oligonucleotide probe oₓ are associated with the gene list for the particular subblock.

20. A method according to claim 8 wherein:
(i) each oligonucleotide probe assigned to a subblock has a degeneracy value indicating the number of different genes that hybridize to that oligonucleotide probe; and
(ii) the steps (c)-(d) are iteratively repeated until for each oligonucleotide probe oₓ assigned to the particular subblock, all genes gₐ that hybridize to the oligonucleotide probe oₓ are associated with the gene list for the particular subblock.

21. A method according to claim 7 in which:
(i) each oligonucleotide probe assigned to the subblock has a degeneracy value indicating the number of different genes that hybridize to that oligonucleotide probe, the degeneracy value being equal to or below a particular threshold T for each oligonucleotide probe assigned to the subblock; and
(ii) each gene gₐ associated with the gene list for the subblock hybridizes to at least one oligonucleotide probe oₓ having a degeneracy value less than the particular threshold T.

22. A method according to claim 1 wherein:
(i) each oligonucleotide probe assigned to a subblock has a degeneracy value indicating the number of different genes that hybridize to that oligonucleotide probe, and (ii) the degeneracy value is equal to or below a particular threshold T for each oligonucleotide probe assigned to the subblock.

23. A method according to claim 22 wherein the particular threshold T is no more than 100.

24. A method according to claim 23 wherein the particular threshold T is no more than 50.

25. A method according to claim 24 wherein the particular threshold T is no more than 20.

26. A method according to claim 25 wherein the particular threshold T is no more than ten.

27. A method according to claim 26 wherein the particular threshold T is no more than five.

28. A method according to claim 27 wherein the particular threshold T is no more than four.

29. A method according to claim 28 wherein the particular threshold T is no more than three.

30. A method according to claim 29 wherein the particular threshold T is no more than two.

31. A method according to claim 30 wherein the particular threshold T is one.

32. A method according to claim 1 in which expression levels are determined for each gene gᵢ that hybridizes to oligonucleotide probes assigned to a particular subblock by a method which comprises solving a system of linear equations for the hybridization of each gene gᵢ to each oligonucleotide probe oⱼ assigned to the particular subblock.

33. A method according to claim 32 wherein the system of linear equations is of the form
\vec{E}=(Ĥ′)⁻¹·\vec{S}′
wherein:
(a) each element Eᵢ of the vector \vec{E} indicates abundance of a nucleotide sequence in the sample corresponding to a particular gene gᵢ;
(b) each element Sⱼ of the vector \vec{S}′ indicates a level of hybridization to a particular oligonucleotide probe oⱼ; and
(c) each element Hᵢⱼ of the matrix Ĥ′ indicates hybridization affinity of the nucleotide sequence corresponding to said particular particular gene gᵢ for the particular oligonucleotide probe oⱼ.

34. A method according to claim 1 wherein each of the nucleotide sequences has a length lᵢ equal to the length of the corresponding gene.

35. A method according to claim 1 wherein the length of each different nucleic acid is decreased before hybridization so that each different nucleic acid has a decreased length Lᵢ=lᵢ−ΔLᵢ that is less than the length of the corresponding gene.

36. A method according to claim 35 wherein the length is decreased by enzymatic digestion.

37. A method according to claim 35 wherein the length of each different nucleic acid is decreased, on average, by a controled amount <ΔL>.

38. A method according to claim 37 wherein the amount <ΔL> is between about 50 and about 500 bases.

39. A method according to claim 38 wherein the amount <ΔL> is between about 50-100 bases.

40. A method according to claim 38 wherein the amount <ΔL> is between about 100-500 bases.

41. A method according to claim 35 wherein the length of each different nucleic acid is decreased by a method which comprises:
(i) protecting each nucleic acid along a particular length; and
(ii) removing the unprotected portion.

42. A method according to claim 35 wherein the average decreased length <L> is controled.

43. A method according to claim 42 wherein the average decreased length <L> is less than or equal to about 500 bases.

44. A method according to claim 43 wherein the average decreased length <L> is less than or equal to about 100 bases.

45. A method according to claim 44 wherein the average decreased length <L> is about 50 bases.

46. A method according to claim 42 wherein the average decreased length <L> is between about 50 and 100 bases.

47. A method according to claim 42 wherein the average decreased length <L> is between about 100 and 500 bases.

48. A method for assigning all or some of a plurality of oligonucleotide probes to subblocks suitable for analyzing data from hybridization of a sample to an array of the oligonucleotide probes,
wherein the sample comprises a plurality of nucleotide sequences, each nucleotide sequence corresponding to a particular gene,
which method comprises steps of:
(a) associating a gene gₐ with a gene list for a subblock, wherein the gene gₐ is not already associated with a gene list for a subblock; and
(b) assigning an oligonucleotide probe oₓ to the subblock, wherein the oligonucleotide probe oₓ hybridizes to the gene gₐ,
wherein the steps are repeated for each subblock until each gene is associated with a gene list for a subblock.

49. A method according to claim 48 further comprising steps of:
(c) for each probe oₓ assigned to the subblock, associating genes g_{b} with the gene list for the subblock, wherein each gene g_{b} hybridizes to the probe oₓ; and
(d) for each gene g_{b} associated with the gene list, assigning an oligonucleotide probe o_{y} to the subblock, wherein the oligonucleotide probe o_{y} hybridizes to the gene g_{b}.

50. A method according to claim 49 wherein the steps of:
(c) associating genes g_{b} with the gene list for the subblock; and
(d) assigning an oligonucleotide probe o_{y} for each gene g_{b} associated with the gene list are iteratively repeated.

51. A method according to claim 50 wherein the step (c)-(d) are repeated for not more than 100 iterations.

52. A method according to claim 51 wherein the steps (c)-(d) are repeated for not more than 50 iterations.

53. A method according to claim 52 wherein the steps (c)-(d) are repeated for not more than 20 iterations.

54. A method according to claim 53 wherein the steps (c)-(d) are repeated for not more than ten iterations.

55. A method according to claim 54 wherein the steps (c)-(d) are repeated for not more than five iterations.

56. A method according to claim 55 wherein the steps (c)-(d) are repeated for not more than four iterations.

57. A method according to claim 56 wherein the steps (c)-(d) are repeated for not more than three iterations.

58. A method according to claim 57 wherein the steps (c)-(d) are repeated for not more than two iterations.

59. A method according to claim 50 wherein the steps (c)-(d) are iteratively repeated until, for each oligonucleotide probe oₓ assigned to the particular subblock, all genes gₐ that hybridize to the oligonucleotide probe oₓ are associated with the gene list for the particular subblock.

60. A method according to claim 51 wherein:
(i) each oligonucleotide probe assigned to a subblock has a degeneracy value indicating the number of different genes that hybridize to that oligonucleotide probe; and
(ii) the steps (c)-(d) are iteratively repeated until, for each oligonucleotide probe oₓ assigned to the particular subblock, all genes gₐ that hybridize to the oligonucleotide probe oₓ are associated with the gene list for the particular subblock.

61. A method according to claim 48 in which:
(i) each oligonucleotide rpboe assigned to the subblock has a degeneracy value indicating the number of different genes that hybridize to that oligonucleotide probe, the degeneracy value being equal to or below a particular threshold T for each oligonucleotide probe assigned to the subblock; and
(ii) each gene gₐ associated with the gene list for the subblock hybridizes to at least one oligonucleotide probe oₓ having a degeneracy value less than the particular threshold T.

62. A method according to claim 48 wherein:
(i) each oligonucleotide probe assigned to a subblock has a degeneracy value indicating the number of different genes that hybridize to that oligonucleotide probe, and
(ii) the degeneracy value is equal to or below a particular threshold T for each oligonucleotide probe assigned to the subblock.

63. A method according to claim 48, wherein the array comprises a plurality (N₀) of oligonucleotide probes having a particular sequence length n so that all nucleic acid sequences having the particular sequence length are present on the array.

64. A method for selecting a particular sequence length n for an array comprising a plurality (N₀) of oligonucleotide probes having the particular sequence length n, which method comprises:
(a) identifying a sequence length n providing an average probe degeneracy <d(n)> suitable for analyzing nucleic acid expression using the array; and
(b) selecting the identified sequence length n,
wherein the average probe degeneracy <d(n)> indicates the number of different nucleic acids that hybridize, on average, to a particular oligonucleotide probe.

65. A method according to claim 64 wherein each of the different nucleic acids corresponds to a gene in a plurality(N_{g}) of different genes.

66. A method according to claim 65 wherein each of the nucleotide sequences has a length lᵢ equal to the length of the corresponding gene.

67. A method according to claim 66 wherein the length of each different nucleic acid is decreased before hybridization so that each different nucleic acid has a decreased length Lᵢ=lᵢ−ΔLᵢ that is less than the length of the corresponding gene.

68. A method according to claim 67 wherein the length is decreased by enzymatic digestion.

69. A method according to claim 67 wherein the length of each different nucleic acid is decreased, on average, by a controled amount <ΔL>.

70. A method according to claim 69 wherein the amount <ΔL> is between about 50 and about 500 bases.

71. A method according to claim 70 wherein the amount <ΔL> is between about 50-100 bases.

72. A method according to claim 70 wherein the amount <ΔL> is between about 100-500 bases.

73. A method according to claim 67 wherein the length of each different nucleic acid is decreased by a method which comprises:
(i) protecting each nucleic acid along a particular length; and
(ii) removing the unprotected portion.

74. A method according to claim 67 wherein the average decreased length <L> is controled.

75. A method according to claim 74 wherein the average decreased length <L> is less than or equal to about 500 bases.

76. A method according to claim 75 wherein the average decreased length <L> is less than or equal to about 100 bases.

77. A method according to claim 76 wherein the average decreased length <L> is about 50 bases.

78. A method according to claim 74 wherein the average decreased length <L> is between about 50 and 100 bases.

79. A method according to claim 74 wherein the average decreased length <L> is between about 100 and 500 bases.

80. A method according to claim 64 wherein the nucleic acids hybridize to the oligonucleotide probes with no more than a particular number (m) of base-pair mismatches.

81. A method according to claim 80 wherein the average probe degeneracy
<d(n)> is provided by the relation
$$⟨ d ( n ) ⟩ = \frac{{N}_{g}}{{N}_{0}} ( 1 - n + ⟨ L ⟩ ) \times c$$
c is provided by the relation
$$c = {\sum }_{k = 0}^{m} ( \begin{matrix}n \\ k\end{matrix} ) {3}^{k} ,$$
and
<L> indicates the average length of the different nucleic acids.

82. A method according to claim 64 wherein the average probe degeneracy <d(n)> is provided by the relation
$$⟨ d ( n ) ⟩ = \frac{{N}_{g}}{{N}_{0}} ( 1 - n + ⟨ L ⟩ ) \times c$$
wherein
<L> indicates the average length of the different nucleic acids, and
c indicates the number of the different nucleic acids that hybridize, on average, to an oligonucleotide probe having the particular sequence length n.

83. A method according to claim 64 wherein the step (a) of identifying a sequence length n comprises:
(i) comparing oligonucleotide sequences having a particular sequence length n with sequences of the different nucleic acids, so that nucleic acids which hybridize to each oligonucleotide sequence are identified; and
(ii) determining the average probe degeneracy <d(n)> from the number of different nucleic acids that hybridize to each oligonucleotide sequence.

84. A method according to claim 64 wherein the identified sequence length n provides an average probe degeneracy <d(n)> that is less than or equal to about five.

85. A method according to claim 64 wherein the identified sequence length n provides an average probe degeneracy <d(n)> that is less than or equal to about four.

86. A method according to claim 64 wherein the identified sequence length n provides an average probe degeneracy <d(n)> that is less than or equal to about three.

87. A method according to claim 64 wherein the identified sequence length n provides an average probe degeneracy <d(n)> that is less than or equal to about two.

88. A method according to claim 64 wherein the identified sequence length n provides an average probe degeneracy <d(n)> of about one.

89. A method according to claim 64 wherein the step (a) of identifying a sequence length n comprises:
(i) assigning all or some of a plurality of oligonucleotide probes having a particular sequence length n to subblocks suitable for analyzing data from hybridization of a sample to an array of the oligonucleotide probes; and
(ii) determining the average probe degeneracy <d(n)> from the oligonucleotide probes assigned to the subblocks.

90. A method according to claim 89 wherein the plurality of oligonucleotide probes is a plurality all nucleic acid sequences having the particular length n.

91. A method according to claim 89 wherein the oligonucleotide probes are assigned to subblocks according to a method which comprises steps of:
(a) associating a gene gₐ with a gene list for a subblock, wherein the gene g_{b} is not already associated with a gene list for a subblock; and
(b) assigning an oligonucleotide probe oₓ to the subblock, wherein the oligonucleotide probe oₓ hybridizes to the gene gₐ,
wherein the steps are repeated for each subblock until each gene is associated with a gene list for a subblock.

92. A method according to claim 91 further comprising steps of:
(c) for each probe oₓ assigned to the subblock, associating genes g_{b} with the gene list for the subblock, wherein each gene g_{b} hybridizes to the probe oₓ; and
(d) for each gene g_{b} associated with the gene list, assigning an oligonucleotide probe o_{y} to the subblock, wherein the oligonucleotide probe o_{y} hybridizes to the gene g_{b}.

93. A method according to claim 92 wherein the steps of:
(c) associating genes g_{b} with the gene list for the subblock; and
(d) assigning an oligonucleotide probe o_{y} for each gene g_{b} associated with the gene list
are iteratively repeated.

94. A method according to claim 93 wherein the step (c)-(d) are repeated for not more than 100 iterations.

95. A method according to claim 94 wherein the steps (c)-(d) are repeated for not more than 50 iterations.

96. A method according to claim 95 wherein the steps (c)-(d) are repeated for not more than 20 iterations.

97. A method according to claim 96 wherein the steps (c)-(d) are repeated for not more than ten iterations.

98. A method according to claim 97 wherein the steps (c)-(d) are repeated for not more than five iterations.

99. A method according to claim 98 wherein the steps (c)-(d) are repeated for not more than four iterations.

100. A method according to claim 99 wherein the steps (c)-(d) are repeated for not more than three iterations.

101. A method according to claim 101 wherein the steps (c)-(d) are repeated for not more than two iterations.

102. A method according to claim 93 wherein the steps (c)-(d) are iteratively repeated until, for each oligonucleotide probe oₓ assigned to the particular subblock, all genes gₐ that hybridize to the oligonucleotide probe oₓ are associated with the gene list for the particular subblock.

103. A method according to claim 93 wherein
(i) each oligonucleotide probe assigned to a subblock has a degeneracy value indicating the number of different genes that hybridize to that oligonucleotide probe; and
(ii) the steps (c)-(d) are iteratively repeated until, for each oligonucleotide probe oₓ assigned to the particular subblock, all genes gₐ that hybridize to the oligonucleotide probe oₓ are associated with the gene list for the particular subblock.

104. A method according to claim 91 in which:
(i) each oligonucleotide probe assigned to the subblock has a degeneracy value indicating the number of different genes that hybridize to that oligonucleotide probe, the degeneracy value being equal to or below a particular threshold T for each oligonucleotide probe assigned to the subblock; and
(ii) each gene gₐ associated with the gene list for the subblock hybridizes to at least one oligonucleotide probe oₓ having a degeneracy less than the particular threshold T.

105. A method according to claim 91 wherein:
(i) each oligonucleotide probe assigned to a subblock has a degeneracy value indicating the number of different genes that hybridize to that oligonucleotide probe, and
(ii) the degeneracy value is equal to or below a particular threshold T for each oligonucleotide probe assigned to the subblock.
